(19) United States
(12) Patent Application Publication
(10) Pub. No.: US 2020/0129610 A1
(43) Pub. Date: Apr. 30, 2020

(54) COMPOSITIONS AND METHODS FOR PRODUCTION OF COLD-CHAIN VACCINES

(71) Applicant: University of Southern California, Los Angeles, CA (US)

(72) Inventors: Jae Jung, Los Angeles, CA (US); Daiki Hara, Los Angeles, CA (US); Byeong Chang, Los Angeles, CA (US); Francisca Gbormittah, Los Angeles, CA (US); Michael Reilly, Los Angeles, CA (US); Woo-Jin Shin, Los Angeles, CA (US)

(21) Appl. No.: 16/669,401

(22) Filed: Oct. 30, 2019

Related U.S. Application Data
(60) Provisional application No. 62/753,624, filed on Oct. 31, 2018.

(51) Int. Cl. A61K 39/13; C12N 7/00

(52) U.S. Cl. CPC A61K 39/13; A61K 2039/5252; C12N 7/00

(57) ABSTRACT

This disclosure provides a novel lyophilized formulation that incorporates a surfactant solution to stabilize the Sabin inactivated polio vaccine and demonstrate the vaccine efficacy in an in vivo challenge model. Furthermore, SE-HPLC analysis of D-antigen content in in inactivated polio vaccine can be used to provide a method for high throughput evaluation of inactivated poliovirus stability.

### CROSS-REFERENCE TO RELATED APPLICATIONS

This application claims priority under 35 U.S.C. § 119(e) to U.S. Provisional Application No. 62/753,624, filed Oct. 31, 2018, the content of which is incorporated herein by reference in its entirety.

### BACKGROUND

Throughout this application, several technical publications are referenced by an Arabic numeral. The complete bibliographic citation for each reference is found immediately preceding the claims. The contents of each publication so referenced and the publications referenced within the specification are hereby incorporated into the present disclosure to more fully describe the state of the art to which this invention pertains.

Twenty-nine years following the initiation of the Global Polio Eradication Initiative, the cases of poliomyelitis in the world have come down from 350,000 to 12 cases remaining in Pakistan and Afghanistan. As vaccination coverage continues to improve, the world remains an arms-length away from a global public health victory of eradicating polio. In 2016, the world successfully coordinated the change from trivalent oral polio vaccine (OPV) to bivalent oral polio vaccine. Ability to shed the vaccine onto others, low cost, and easy oral administration championed OPV as the main weapon in vaccinating the world against polio. However, the OPV's ability to shed onto others is accompanied by the virus' ability the mutate and induce vaccine-derived poliovirus (cVDPV), which presents a major threat against the eradication as cases of wild polio infection inch closer to 0. By October of 2017, the world only reported 12 cases of polio, but Syria and Democratic Republic of Congo saw a combined 62 cases of cVDPV. Thus, the removal of trivalent OPV will continue as bivalent OPV will eventually shift to monovalent, and finally to Inactivated Polio Vaccine (IPV), an intramuscularly injectable vaccine against poliovirus. However, another shift is imminent as Sabin Inactivated Polio Vaccine (sIPV) takes over IPV in order to eliminate the chances of wild poliovirus reintroduction from IPV production factories. Currently, Japan and China have lead the path with their distribution of commercial sIPV stocks.

With IPV becoming a major focus in the path to global polio eradication, its disadvantages are clearly highlighted. One major issue affecting IPV is the high sensitivity to the cold chain, a process of maintaining 2-8° C. temperature of a drug through its lifetime from production to transport to administration. Millions of vaccine doses are wasted annually due to complications with the cold chains even as vaccine vial monitors, colored patches to indicate vaccine vials overexposure to heat, currently help to reduce injections of destroyed vaccines. Thus, major effort has gone into removing the cold chain from multiple vaccines to reduce cost and ensure the safe injection of vaccines. In the case of polio, extensive study has developed new vaccines that are not sensitive to the cold chain, but many of these options do not fit requirements to inject in people or can only induce high immunogenicity when used as booster. Among the vaccine stabilization efforts, the most common method to stabilize vaccines in the past has been a freeze dying process known as lyophilization. The lyophilization process removes water from a liquid drug by freezing and inducing the frozen water to sublimate under low pressure conditions. In the past, IPV has presented multiple complications with producing an injectable lyophilized polio vaccine. Historically, IPV has been difficult vaccine to lyophilize due to low recovery following lyophilization and low stability over time at ambient temperatures. Moreover, a recent study successfully lyophilized IPV with high recovery, but the moisture content was beyond European Pharmacopeia standards.

As the world approaches the eradication of polio in the next few years, the final steps to eradicate polio include the switch from oral polio vaccines to inactivated polio vaccines (IPV) and eventually to Sabin inactivated polio vaccines. Each switch will help to ensure poliovirus cannot reemerge from production lines or vaccine mutations, but each switch introduces a new list of challenges. A major challenge facing inactivated polio vaccines is their need to remain refrigerated. The burden to maintain IPV within the cold chain transport requires enormous amount of resources and millions of doses are still wasted annually. Thus, taking IPV out of the cold chain allows vaccine campaigns to remove a huge barrier towards maintaining global vaccination coverage. Recent advances in lyophilization have helped multiple vaccines find a temperature stable formulation. However, polio vaccines remain yet to capture a stable, safe formula for lyophilization, which maintains antigenicity following lyophilization.

Thus, a need exists in the art to provide a process to produce a vaccine formulation that overcomes these limitations, as well as the vaccines produced by these methods.

### SUMMARY OF THE DISCLOSURE

In one aspect, this disclosure provides a novel lyophilized formulation that incorporates a surfactant solution to stabilize the Sabin inactivated polio vaccine and demonstrate the vaccine efficacy in an in vivo challenge model. Furthermore, SE-HPLC analysis of D-antigen content in in inactivated polio vaccine can be used to provide a method for high throughput evaluation of inactivated poliovirus stability.

Thus, in one aspect, provided herein a vaccine formulation having a pH of from about 5.5 to about 8.5, the comprising, consisting essentially of, or yet further consisting of:
a. an effective amount of a virus or viral antigen;
b. from about 2% to about 10% of a bulking agent;
c. from about 5 mM to about 25 mM of a buffer;
d. from about 0.5% to about 1.5% of a sugar composition; and
e. from about 0.1% to about 10% or from about 0.5 mM to about 1.5 mM of a stabilizer.

In one aspect, the vaccine formulation further comprises, or consists essentially of, or yet further consists of, from about 0.005% to about 1.5% of a surfactant solution. In another aspect, the vaccine formulation further comprises, or consists essentially of, or yet further consists of, from about 0.5 mM to about 1.5 mM of a metal ion.

In one embodiment, the bulking agent is from about 2% to about 10% of Mannitol or Glycine, or an equivalent of each thereof. In a further aspect, the bulking agent is from about 4.5% to about 5.5% of Mannitol or from about 2.0% to about 3.0% of Glycine, or an equivalent of each thereof. In another aspect, the buffer is from about 5 mM to about 25 mM of Histidine or from about 5 mM to about 25 mM of Tris-HCl, or an equivalent of each thereof. In one aspect, the buffer in the composition is from about 9 mM to about 11 mM of Histidine or from about 9 mM to about 11 mM of Tris-HCl, or an equivalent of each thereof.

In another embodiment, the vaccine formulation comprises from about 0.5% to about 1.5% of sugar. In another aspect, the sugar is from about 0.75% to about 1.25% of Sucrose or Sorbitol, or an equivalent of each thereof.

In one embodiment, the surfactant in the vaccine is from about 0.005% to about 1.5% of a surfactant solution comprises of Polysorbate 20, Polysorbate 80, or Poloxamer 188 (Pluronic F68), or an equivalent of each thereof.

In a further aspect, the surfactant comprises, or consists essentially of, or yet further consists of a surfactant solution from the group of: from about 0.005% to about 1% of Polysorbate 20; from about 0.005% to about 1% of Polysorbate 80, or from about 0.05% to about 1.5% of Pluronic F68, or an equivalent of each thereof.

In one embodiment, the metal ion comprises, or consists essentially of, or yet further consists of, from about 0.5 mM to about 1.5 mM of MgSO₄ or MgCl₂, or an equivalent of each thereof. In one embodiment, the metal ion comprises from about 0.8 mM to about 1.2 mM of MgSO₄, or an equivalent thereof.

In one aspect, stabilizer in the vaccine comprises, or consists essentially of, or yet further consists of, from about 0.1% to 10% of Polyvinylpyrrolidone or Polyethylene glycol or from about 0.5 mM to about 1.5 mM of MgSO₄, or an equivalent of each thereof. In another aspect, the stabilizer in the vaccine is from about 0.8% to about 1.2% of Polyvinylpyrrolidone or Polyethylene glycol or from about 0.8 mM to about 1.2 mM of MgSO₄, or an equivalent of each thereof.

In another aspect, the formulation is at a pH of from about 5.5 to about 8.5.

In a further aspect, the virus or viral antigen in the vaccine comprises, or consists essentially of, or yet further consists of, an effective amount of D-antigen units of formaldehyde-inactivated polio virus or viral antigen. Additional viral antigens that can be used in the vaccine formulation include for example, measles virus, typhoid viral antigents, Meningococcal polysaccharide vaccine Groups A and C. In one embodiment, the virus or viral antigen comprises, or consists essentially of, or yet consists of, an effective amount of D-antigen units of a heat-inactivated polio virus or viral antigen, e.g., wherein the effective amount of the formaldehyde-inactivated polio virus is from about 0.3×10⁷ to about 10.0×10⁷ pfu/ml; or wherein the effective amount of the heat-inactivated polio virus is from about 0.6×10⁷ to about 7.0×10⁷ pfu/ml; or alternatively wherein the effective amount of the vaccine or antigen comprises from about 20 D-antigen Units/ml to about 100 D-antigen Units/ml; or alternatively wherein the effective amount of the virus or viral antigen comprises about 80 D-antigen Units/ml.

Yet further provided is a vaccine formulation as set forth in Table 3. In one aspect, the effective amount of the vaccine or antigen comprises at least about 15% D-antigen recovery or an equivalent thereof or wherein the effective amount of the vaccine or antigen comprises at least about 15% D-antigen recovery as measured using size-exclusion high-performance liquid chromatography (SE-HPLC) or an equivalent thereof.

In one aspect, the effective amount of the vaccine or antigen comprises at least about 15% D-antigen recovery as measured using enzyme-linked immunosorbent assay (ELISA) or an equivalent thereof.

Also provided herein is a lyophilized formulation of the vaccine formulation of any one of vaccine formulations as described herein. In one aspect, the vaccine the lyophilized vaccine formulation has a moisture content of no more than about 2% water or an equivalent thereof.

Also provided herein is a composition comprising an effective amount of the lyophilized formulation of as described herein and a pharmaceutically acceptable carrier.

Yet further provided is a kit comprising one or more of the vaccine formulation as described herein and instructions for use.

Further provided herein is a method to immunize a subject against a viral infection comprising, or consisting essentially of, or yet further consisting of administering to a subject in need thereof an effective amount of the vaccine composition as described herein, and wherein the subject is optionally a mammal or a human subject, further optionally an infant or juvenile. The vaccine can be administered in one or more doses.

Also provided herein is a method to prepare a vaccine formulation, the method comprising, or alternatively consisting essentially of, or yet further consisting of, admixing:
a. an effective amount of a virus or viral antigen;
b. from about 2% to about 10% of a bulking agent;
c. from about 5 mM to about 25 mM of a buffer;
d. from about 0.5% to about 1.5% of a sugar composition; and
e. from about 0.1% to about 10% or from about 0.5 mM to about 1.5 mM of a stabilizer.

In one aspect of the method, it further comprises admixing from about 0.005% to about 1.5% of a surfactant solution with the other components.

In one aspect, the method further comprises admixing from about 0.5 mM to about 1.5 mM of a metal ion with the other components.

In one aspect of the method, the bulking agent is from about 2% to about 10% of Mannitol or Glycine, or an equivalent of each thereof. In another aspect, the bulking agent is from about 4.5% to about 5.5% of Mannitol or from about 2.0% to about 3.0% of Glycine, or an equivalent of each thereof. In a further aspect, the buffer is from about 5 mM to about 25 mM of Histidine or from about 5 mM to about 25 mM of Tris-HCl, or an equivalent of each thereof. In a yet further aspect, the buffer is from about 9 mM to about 11 mM of Histidine or from about 9 mM to about 11 mM of Tris-HCl, or an equivalent of each thereof. In another embodiment, the sugar is from about from about 0.5% to about 1.5% of Sucrose or Sorbitol, or an equivalent of each thereof. In a further aspect, the sugar is from about 0.75% to about 1.25% of Sucrose or Sorbitol, or an equivalent of each thereof.

In one embodiment, the surfactant solution comprises, or consists essentially of, or yet further consists of from about 0.005% to about 1.5% of a surfactant solution from the group of: Polysorbate 20, Polysorbate 80, Poloxamer 188 (Pluronic F68), or an equivalent of each thereof. In one aspect, the surfactant solution comprises, or consists essentially of, or yet further consists of, a solution from the group of: from about 0.005% to about 1% of Polysorbate 20, from about 0.005% to about 1% of Polysorbate 80, from about 0.05% to about 1.5% of Pluronic F68, or an equivalent of each thereof.

In one embodiment, the metal ion in the method comprises, or consists essentially of, or yet further consists of from about 0.5 mM to about 1.5 mM of MgSO₄ or MgCl₂, or an equivalent of each thereof. In an alternative aspect, the metal ion in the method comprises, from about 0.8 mM to about 1.2 mM of MgSO₄, or an equivalent thereof. In another aspect of the method, the stabilizer is from about 0.1% to 10% of Polyvinylpyrrolidone or Polyethylene glycol or from about 0.5 mM to about 1.5 mM of MgSO₄, or an equivalent of each thereof. In another aspect of the method, the stabilizer from about 0.8% to about 1.2% of Polyvinylpyrrolidone or Polyethylene glycol or from about 0.8 mM to about 1.2 mM of MgSO₄, or an equivalent of each thereof. In a further aspect of the method, the final formulation is at a pH from about 5.5 to about 8.5.

In one embodiment of the method, the virus or viral antigen comprises an effective amount of D-antigen units of formaldehyde-inactivated polio virus or viral antigen, or alternatively an effective amount of D-antigen units of a heat-inactivated polio virus or viral antigen, or yet further, the effective amount of the formaldehyde-inactivated polio virus is from about 0.3×10⁷ to about 10.0×10⁷ pfu/ml. In one aspect of the method, the effective amount of the heat-inactivated polio virus is from about 0.6×10⁷ to about 7.0×10⁷ pfu/ml, or alternatively the effective amount of the vaccine or antigen comprises from about 20 D-antigen Units/ml to about 100 D-antigen Units/ml, or yet further the effective amount comprises about 80 D-antigen Units/ml.

In another aspect of this disclosure, provided herein is a method comprising admixing the components as set forth in Table 3 and adjusting the pH to from about 5.5 to about 8.5. In one aspect of this method, the effective amount of the vaccine or antigen comprises at least about 15% D-antigen recovery or an equivalent thereof. In an alternative embodiment of this method, it further comprises using size-exclusion high-performance liquid chromatography (SE-HPLC) to screen for the effective amount of the vaccine or antigen comprising of at least about 15% D-antigen recovery, or an equivalent thereof following lyophilization. In a further aspect, the method further comprises using enzyme-linked immunosorbent assay (ELISA) to screen for the effective amount of the vaccine or antigen comprising of at least about 15% D-antigen recovery, or an equivalent thereof following lyophilization. In a yet further aspect of the method, it comprises lyophilizing the vaccine formulation.

In one aspect of the disclosed methods and compositions, the lyophilized vaccine formulation has a moisture content of at most about 2% water, or an equivalent thereof.

Also provided herein is a vaccine formulation prepared or obtained by a method as disclosed herein. These vaccine can be used in a method to immunize a subject against a viral infection comprising administering to a subject in need thereof an effective amount of the vaccine formulation, and wherein the subject is optionally a mammal or a human subject, further optionally an infant or juvenile.

### BRIEF DESCRIPTION OF THE FIGURES

FIG. 1: Purified inactivated Sabin Poliovirus. Polioviral particles are comprised of icosahedral capsid proteins that consist of VP1, VP2, VP3, and VP4. To check the purity of Sabin inactivated poliovirus, virions were separated on SDS-PAGE, followed by silver staining. Lane 1, molecular weight standards; lane 2, before tangential flow filtration (TFF); lane 3, after TFF; lane 4, after size exclusion chromatography (SEC); lane 5, after ion exchange chromatography (IEC).

FIGS. 2A-2B: SE-HPLC reliably measures D-antigen stability of sIPV. Poliovirus antigens are divided into D-antigen (D-Ag) and C-antigen (C-Ag) where only D-Ag shows major immunogenicity. Size-exclusion high-performance liquid chromatography (SE-HPLC) was used as a novel method for determining the antigenicity of sIPV through separation of intact viral particles from disintegrated capsid proteins based on hydrodynamic radius. (FIG. 2A): sIPV chromatogram detecting 336 nm emission from 280 nm excitation through SE-HPLC. (FIG. 2B): SE-HPLC chromatograms of sIPV main peak eluate after one-week storage at 4° C. in liquid state.

FIGS. 3A-3C: Lyophilized sIPV remains stable at elevated temperatures. To test the thermostability of lyophilized sIPV, lyophilized sIPV from the formulation F4, F8 and F9 was incubated at different temperatures and measured D-Ag recovery using conventional ELISA. (FIG. 3A): D-Ag unit recovery over 4 weeks of incubation in 4° C. (FIG. 3B): D-antigen unit recovery over 4 weeks of incubation in 25° C. (FIG. 3C): D-antigen unit recovery over 4 weeks of incubation in 40° C.

FIGS. 4A-4B: Lyophilized sIPV effectively protects mice against wild-type poliovirus challenge. (FIG. 4A): Mean neutralization antibody titers of vaccination group. Blood of vaccinated cPVR transgenic mice (n=8) with commercial IPOL-IPV, sIPV or reconstituted lyophilized sIPV incubated in either 4° C. or 37° C. for 4 weeks was collected at day 21 to measure neutralizing antibody titers against 27 100TCID50 of Sabin type 1 poliovirus. For the statistical analysis, one-way ANOVA Kruskal-Wallis test was used. *P<0.05, ***P<0.001. (FIG. 4B): In vivo vaccine efficacy of lyophilized IPV. To investigate the protective efficacy of thermbostabilized sIPV in vivo, cPVR transgenic mice (n=8) were vaccinated and boosted with commercial IPOL-IPV, sIPV or reconstituted lyophilized sIPV incubated in either 4° C. or 37° C. for 4 weeks. These mice were then challenged at day 28 with wild-type PV Mahoney strain to test virus-induced paralysis. Commercial IPOL-IPV (trivalent polio vaccine distributed by Sanofi Pasteur) was used as a control.

FIG. 5: Depicts the results of a study wherein the benefit of additional stabilizer was evaluated by determining the recovery of virus after lyophilization. All tested formulations contain the composition of the control formulation at 5% mannitol, 1% sucrose, 1 mM MgSO4, 0.01% polysorbate 20, in 10 mM histidine at pH 7.0. Pre-lyo sample is the sample without the lyophilization. Addition of 1% PEG (polyethylene glycol) or 1% PVP (Polyvinylpyrrolidone or Povidone) improved the recovery from 80% of the control to 95-98%, respectively.

FIG. 6: Production scheme of lyophilized Sabin inactivated poliovirus.

FIG. 7: Comparison between D-Antigen ELISA and SE-HPLC peaks. The relative potency of the main peak and post peak was measured by dividing the ELISA titer of each peak by the area under the UV peaks.

FIGS. 8A-8B: (FIG. 8A). D-antigen area from SE-HPLC of static and agitated samples. (FIG. 8B). Percent D-antigen unit recovery after agitation with different surfactants.

FIG. 9: Timeline for in vivo survival study of cPVR mice.

FIG. 10: Determination of optimal time for testing neutralizing antibody titer. Blood of vaccinated cPVR transgenic mice (n=8) sIPV was collected at days 13 and 21 to measure neutralizing antibody titers against 100TCID₅₀ of Sabin type 1 poliovirus. Commercial IPOL-IPV was included as a control.

### DETAILED DESCRIPTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

“An effective amount” refers to the amount of the defined component sufficient to achieve the desired chemical composition or the desired biological and/or therapeutic result. That result can be the desired pH or chemical or biological characteristic, e.g., stability of the formulation. In other aspects, the desired result is the alleviation of the signs, symptoms, or causes of a disease, or any other desired alteration of a biological system. When the desired result is a therapeutic response, the effective amount will vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art.

A “subject” of diagnosis or treatment is a prokaryotic or a eukaryotic cell, a tissue culture, a tissue or an animal, e.g., a mammal, including a human. Non-human animals subject to diagnosis or treatment include, for example, a human patient, a simian, a murine, a canine, a leporid, such as a rabbit, livestock, sport animals, and pets.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

As used herein, to “treat” further includes systemic amelioration of the symptoms associated with the pathology and/or a delay in onset of symptoms. Clinical and sub-clinical evidence of “treatment” will vary with the pathology, the individual and the treatment.

A formulation of the present invention can be administered by any suitable route, specifically by parental (including subcutaneous, intramuscular, intravenous and intradermal) administration. It will also be appreciated that the preferred route will vary with the condition and age of the recipient, and the disease being treated. Methods of determining the most effective means and dosage of administration are known to those of skill in the art and will vary with the composition used for therapy, the purpose of the therapy, and the subject being treated. Single or multiple administrations can be carried out with the dose level and pattern being selected by the treating physician. Suitable dosage formulations and methods of administering the agents are known in the art.

The formulations of the present invention can be used in the manufacture of medicaments and for the treatment of humans and other animals by administration in accordance with conventional procedures.

An “isoelectric point” or “pI” refers to the pH at which an amphoteric molecule, such as an antibody, carries no net electrical charge. An amphoteric molecule contains both positive and negative charges depending on the functional groups present in the molecule. The net charge on the molecule is affected by pH of the surrounding environment and can become more positively or negatively charged due to the loss or gain of protons (H+). The pI is the pH value at which the molecule carries no electrical charge or the negative and positive charges are equal. The pI value can affect the solubility of a molecule at a given pH. Such molecules have minimum solubility in water or salt solutions at the pH which corresponds to their pI and often precipitate out of solution. Biological amphoteric molecules such as proteins contain both acidic and basic functional groups. Amino acids which make up proteins may be positive, negative, neutral or polar in nature, and together give a protein its overall charge. At a pH below their pI, proteins carry a net positive charge; above their pI they carry a net negative charge. A “theoretical pI” of a protein depends on the amino acid composition of the molecule and can be calculated with methods known in the art, e.g., with a script available on the website of biopython.org/wiki/Main_Page, last accessed Sep. 2, 2009.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, canine, bovine, porcine, murine, rat, avian, reptilian and human.

“Prokaryotic cells” that usually lack a nucleus or any other membrane-bound organelles and are divided into two domains, bacteria and archaea. Additionally, instead of having chromosomal DNA, these cells' genetic information is in a circular loop called a plasmid. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to bacillus bacteria, E. coli bacterium, and Salmonella bacterium.

As used herein, the term “label” intends a directly or indirectly detectable compound or composition that is conjugated directly or indirectly to the composition to be detected, e.g., polynucleotide or protein such as an antibody so as to generate a “labeled” composition. An antibody in a formulation or in a coformulation with other coformulated antibodies can be labeled to facilitate detection or stability analysis. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, the term “excipient” refers to an inert substance which is commonly used as a diluent, vehicle, preservative, binder, or stabilizing agent for drugs and includes, but is not limited to, proteins (e.g., serum albumin, etc.), amino acids (e.g., aspartic acid, glutamic acid, lysine, arginine, glycine, histidine, alanine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). Also see Remington's Pharmaceutical Sciences (by Joseph P. Remington, 18th ed., Mack Publishing Co., Easton, Pa.) and Handbook of Pharmaceutical Excipients (by Raymond C. Rowe, 5th ed., APhA Publications, Washington, D.C.) which are hereby incorporated in its entirety. Preferably, the excipients impart a beneficial physical property to the formulation, such as increased protein stability, increased protein solubility and decreased viscosity.

The term “buffer” as used herein denotes a pharmaceutically acceptable excipient, which stabilizes the pH of a pharmaceutical preparation. Suitable buffers are well known in the art and can be found in the literature. Pharmaceutically acceptable buffers comprise but are not limited to histidine-buffers, citrate-buffers, succinate-buffers and phosphate-buffers. Independently from the buffer used, the pH can be adjusted at a value from about 5.5 to about 8.5 or alternatively from about 6.5 to about 8.5 or alternatively about 7.0 with an acid or a base known in the art, e.g., succinic acid, hydrochloric acid, acetic acid, phosphoric acid, sulfuric acid and citric acid, sodium hydroxide and potassium hydroxide. Suitable buffers include, without limitation, histidine, histidine buffer, 2-morpholinoethanesulfonic acid (MES), cacodylate, phosphate, acetate, succinate, and citrate.

“Cryoprotectants” are known in the art and include without limitation, e.g., sucrose, trehalose, and glycerol. A cryoprotectant exhibiting low toxicity in biological systems is generally used.

As used herein, the term “surfactant” refers to a pharmaceutically acceptable organic substance having amphipathic structures; namely, it is composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials. In some embodiments of the pharmaceutical formulations described herein, the amount of surfactant is described as a percentage expressed in weight/volume percent (w/v %). Suitable pharmaceutically acceptable surfactants include but are not limited to the group of polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), or sodium dodecyl sulphate (SDS). Polyoxyethylenesorbitan-fatty acid esters include polysorbate 20, (sold under the trademark Tween 20™) and polysorbate 80 (sold under the trademark Tween 80™). Polyethylene-polypropylene copolymers include those sold under the names Pluronic® F68 or Poloxamer 188™. Polyoxyethylene alkyl ethers include those sold under the trademark Brij™. Alkylphenolpolyoxyethylene ethers include those sold under the tradename Triton-X. When polysorbate 20 (Tween 20™) and polysorbate 80 (Tween 80™).

A “lyoprotectant” refers to a pharmaceutically acceptable substance that stabilizes a protein during lyophilization (the process of rapid freezing and drying in a high vacuum). Examples of lyoprotectants include, without limitation, sucrose, trehalose or mannitol.

A “preservative” is a natural or synthetic chemical that is added to products such as foods, pharmaceuticals, paints, biological samples, wood, etc. to prevent decomposition by microbial growth or by undesirable chemical changes. Preservative additives can be used alone or in conjunction with other methods of preservation. Preservatives may be antimicrobial preservatives, which inhibit the growth of bacteria and fungi, or antioxidants such as oxygen absorbers, which inhibit the oxidation of constituents. Common antimicrobial preservatives include, benzalkonium chloride, benzoic acid, cholorohexidine, glycerin, phenol, potassium sorbate, thimerosal, sulfites (sulfur dioxide, sodium bisulfate, potassium hydrogen sulfite, etc.) and disodium EDTA. Other preservatives include those commonly used in patenteral proteins such as benzyl alcohol, phenol, m-cresol, chlorobutanol or methylparaben.

The term “stable formulation” as used herein in connection with the formulations according to the invention denotes a formulation, which preserves its physical stability/identity/integrity and/or chemical stability/identity/integrity and/or biological activity during manufacturing, storage and application. Various analytical techniques for evaluating protein stability are available in the art and reviewed in Reubsaet et al. (1998) J. Pharm. Biomed. Anal. 17(6-7): 955-78 and Wang (1999) Int. J. Pharm. 185(2): 129-88. Stability can be evaluated by storage at selected climate conditions for a selected time period, by applying mechanical stress such as shaking at a selected shaking frequency for a selected time period, by irradiation with a selected light intensity for a selected period of time, or by repetitive freezing and thawing at selected temperatures. The stability may be determined by at least one of the methods selected from the group consisting of visual inspection, SDS-PAGE, IEF, (high pressure) size exclusion chromatography (HPSEC), RFFIT, and kappa/lambda ELISA.

An vaccine “retains its biological activity” in a pharmaceutical formulation, if the biological activity of the antibody at a given time is between about 50% and about 200%, or alternatively between about 60% and about 170%, or alternatively between about 70% and about 150%, or alternatively between about 80% and about 125%, or alternatively between about 90% and about 110%, of the biological activity exhibited at the time the pharmaceutical formulation was prepared as determined.

The term “D-antigen recovery” as used herein in connection with the formulations according to the invention denotes recovery of D-antigen units after lyophilization as measured by SE-HPLC or ELISA or an equivalent thereof.

The term “moisture content” as used herein in connection with the formulations according to the invention denotes the moisture or water content in solid lyophilized formulations as determined by coulometric titration using Karl Fischer (KF) analysis or an equivalent thereof.

### Modes for Carrying Out the Disclosure

Provided herein is a formulation to lyophilize Sabin inactivated polio vaccine. Various surfactants were combined with Sabin inactivated polio vaccine to significantly increase recovery of antigenicity following lyophilization. Formulations containing surfactants achieved recoveries as high as 95%. Furthermore, the lyophilized vaccines remain stable enough after three week incubation in 37° C. to induce anti-poliovirus antibodies protecting transgenic mice against poliovirus infection. To achieve this, a novel size exclusion high performance liquid chromatography based approach was used to ensure sIPV stability. Based on these methods, provided herein are formulations for vaccine stabilizaton, such as sIPV stabilization, that generate high recovery from lyophilization, stability at elevated temperatures, and low residual moisture content. While the experimental results focus on type 1 poliovirus because it is the remaining wild type polio serotype in the world, and type 1 poliovirus vaccine typically results in the most challenging for temperature stability, and this approach is applicable to other vaccine formulations as well.

Vaccine Formulations and their Uses

This disclosure provides novel vaccine formulations. In one aspect, the vaccine formulation has a pH of from about 5.5 to about 8.5, comprising, or consisting essentially of, or yet further consisting of:
a. an effective amount of a virus or viral antigen;
b. from about 2% to about 10% of a bulking agent;
c. from about 5 mM to about 25 mM of a buffer;
d. from about 0.5% to about 1.5% of a sugar composition; and
e. from about 0.1% to about 10% or from about 0.5 mM to about 1.5 mM of a stabilizer.

The formulations can further comprise from about 0.005% to about 1.5% of a surfactant solution.

The formulations can further comprise from about 0.5 mM to about 1.5 mM of a metal ion.

The formulations can further comprise a preservative, lyoprotectant and/or a cryoprotectant.

In one aspect, the bulking agent is from about 2% to about 10% of the bulking or an equivalent thereof. In one aspect, the bulking agent is Mannitol or Glycine, or an equivalent of each thereof, and an equivalent of Mannitol or Glycine intends other known bulking agents that provide the same or similar effectiveness and stability of the above-noted formulation. In another aspect the bulking agent such as Mannitol or Glycine, or an equivalent of each thereof, is provided in an amount from about 3% to about 9%, or alternatively from about 4% to about 8%, or alternatively from about 3% to about 8%, or alternatively from about 3% to about 7%, or alternatively from about 4% to about 7%, or alternatively from about 4% to about 6%, or alternatively about 5%. In one aspect, the bulking agent is about 5% of Mannitol or Glycine, or an equivalent of each thereof. In one aspect, the bulking agent is about 2.5% of Mannitol or Glycine, or an equivalent of each thereof.

In another aspect, the buffer in the formulation is from about 5 mM to about 25 mM, or alternatively from about 5 mM to about 20 mM, or from about 10 mM to about 20 mM, or from about 15 mM to about 25 mM, or from about 15 mM to about 20 mM, or from about 5 mM to about 10 mM, or from 8 mM to about 12 mM, or about 10 mM. In one aspect the buffer comprises or is Histidine or Tris-HCl, or an equivalent of each thereof. An equivalent of Histidine or Tris-HCl intends other known buffering agents that provide the same or similar effectiveness and stability of the above-noted formulation comprising Histidine or Tris-HCl. In one aspect, the buffer is about 10 mM of Histidine or Tris-HCl, or an equivalent of each thereof.

In another aspect, the vaccine formulation comprises a sugar at about 0.5% to about 1.5%, or alternatively from about 0.7% to about 1.5%, or alternatively from about 0.9% to about 1.5%, or alternatively from about 1.0% to about 1.5%, or alternatively from about 0.5% to about 1.2%, or alternatively from about 0.7% to about 1.2%, or alternatively from about 0.9% to about 11%, or alternatively about 1.0%. In one aspect the sugar is Sucrose or Sorbitol, or an equivalent of each thereof. An equivalent of Sucrose or Sorbitol intends other known sugars provided in vaccine formulations that provide the same or similar effectiveness and stability of the above-noted formulation.

In a further aspect, the vaccine formulation comprises from about 0.005% to about 1.5% of a surfactant solution. Alternatively, the vaccine formulation comprises from about 0.01% to about 0.5%, or about 0.01% to about 0.5%, or about 0.01% to about 0.3%, or about 0.01% to about 1.0%, or about 0.5% to about 1.5%, or about 0.001% to about 0.007%, or about 0.001% to about 0.003%, or about 0.001% to about 0.05%, or about 0.1%, of a surfactant solution. In one aspect, the surfactant solution comprises of polysorbate 20, polysorbate 80, or poloxamer 188 (pluronic F68). In a further aspect, the surfactant comprises 0.01% of polysorbate 20.

In a yet further aspect, the formulation comprises 0.5 mM to about 1.5 mM of a metal ion, or from about 0.5 mM to about 1.3 mM, or from about 0.7 mM to about 1.5 mM, or from about 0.9 mM to about 1.5 mM, or from about 0.7 mM to about 1.2 mM, or from about 0.8 mM to about 11 mM, or about 0.9 mM to about 1.1 mM, or about 1 mM of the metal ion. In one aspect the metal ion comprises from about 0.5 mM to about 1.5 mM of MgSO₄ or MgCl₂, or an equivalent or each thereof. In a further aspect, the metal ion comprises about 1 mM of MgSO₄, or an equivalent thereof. An equivalent of MgSO₄ or MgCl₂ intends other known metal ions used in vaccine formulations that provide the same or similar effectiveness and stability of the above-noted formulation.

In another aspect, the vaccine formulation comprises from about 0.1% to 10% of a stabilizer, or from about 0.5% to about 8%, or from about 0.5% to about 8%, or from about 0.5% to about 8%, or from about 0.5% to about 8%, or from about 0.5% to about 5%, or from about 0.5% to about 3%, or about 1% of the stabilizer. In one aspect the stabilizer comprises polyvinylpyrrolidone or polyethylene glycol, or an equivalent thereof. An equivalent of polyvinylpyrrolidone or polyethylene glycol intends other known vaccine stabilizers used in vaccine formulations that provide the same or similar effectiveness and stability of the above-noted formulation. In a further aspect, the stabilizer comprises from about 1% of polyvinylpyrrolidone or an equivalent thereof. In another aspect, the vaccine formulation comprises from about 0.5 mM to about 1.5 mM of a stabilizer. In a further aspect, the stabilizer comprises from about 0.5 mM to about 1.5 of MgSO₄, or alternatively about 1 mM of MgSO₄ or an equivalent thereof. An equivalent of MgSO₄ intends other known vaccine stabilizers used in vaccine formulations that provide the same or similar effectiveness and stability of the above-noted formulation.

The pH of the formulation should be from about 5.5 to about 8.5, or from about 6.1 to about 7.5, or from about 6.2 to about 7.5, or from about 6.3 to about 7.5, or from about 6.4 to about 7.5, or from about 6.4 to about 7.5, or from about 6.5 to about 7.5, or from about 6.6 to about 7.5, or from about 6.7 to about 7.5, or from about 6.8 to about 7.5 or from about 6.9 to about 7.5, or from about 6.2 to about 7.4, or from about 6.3 to about 7.4, or from about 6.4 to about 7.3, or from about 6.4 to about 7.2, or from about 6.5 to about 7.2, or from about 6.8 to about 7.2, or from about 6.6 to about 7.1, or from about 6.9 to about 7.4, or from about 6.9 to about 7.3, or from about 6.9 to about 7.2, or from about 6.9 to about 7.2, or about 7.0.

In one aspect, the above noted formulations are provided without a virus or viral antigen. However when they do comprise the virus or viral antigen, the formulations are suitable for use in formulation of other known vaccine components, e.g., DNA or RNA-viral vaccines, and for the prevention of disease, examples of which can be found on the World Health Organization website. Non-limiting examples of such include, diphtheria, Hepatitis B, Heamohilus influenza type B, Human papillomavirus, influenza, measles, mumps, pertussis, Rubella, Pneumococcal diseases, poliomyelitis (polio), Rotavirus, Tuberculosis, Tetnus, Varicella, Cholera, Hepatitis A, Hepatitis B, Japanese encephalitis, Meningococcal disease, Rabies, Tick-born encephalitis, Typhoid fever, Yellow fever, Dengue, Enterovirus 71, HIV-1, Leishmaniasis Disease, Malaria, Shigella and Ebola.

In one particular aspect, the virus comprises formaldehyde-inactivated polio virus. In another aspect, the virus or viral antigen comprises D-antigen units of a heat-inactivated polio vaccine.

In one aspect, the effective amount of the formaldehyde-inactivated polio virus is from about 0.3×10⁷ to about 10.0×10⁷ pfu/ml, or from about 0.5×10⁷ to about 10.0×10⁷ pfu/ml, or from about 0.5×10⁷ to about 8.0×10⁷ pfu/ml, or from about 0.5×10⁷ to about 6.0×10⁷ pfu/ml, or from about 0.4×10⁷ to about 10.0×10⁷ pfu/ml, or from about 0.5×10⁷ to about 10.0×10⁷ pfu/ml, or from about 0.5×10⁷ to about 9.0×10⁷ pfu/ml, or from about 0.8×10⁷ to about 10.0×10⁷ pfu/ml, or from about 0.5×10⁷ to about 9.0×10⁷ pfu/ml, or about 0.6, or about 0.7, or about 0.8, or about 0.9, or about 1.0, or about 1.5, or about 2.0, or about 3.5, or about 4.0, or about 4.5, or about 5.0, or about 5.5, or about 6.0, each as X 10⁷ pfu/ml. In one aspect, the effective amount of the heat-inactivated polio virus is from about 0.6×10⁷ to about 7.0×10⁷ pfu/ml.

In a further aspect, when the viral antigen comprises D-antigen units of a heat-inactivated polio vaccine, the effective amount of the vaccine or antigen comprises from about 20 D-antigen Units/ml to about 100 D-antigen Units/ml, or from about 30 D-antigen Units/ml to about 90 D-antigen Units/ml, 40 D-antigen Units/ml to about 100 D-antigen Units/ml, or from about 50 D-antigen Units/ml to about 100 D-antigen Units/ml, or from about 60 D-antigen Units/ml to about 100 D-antigen Units/ml, or from about 70 D-antigen Units/ml to about 100 D-antigen Units/ml, or from about 70 D-antigen Units/ml to about 90 D-antigen Units/ml, or from about 75 D-antigen Units/ml to about 85 D-antigen Units/ml, or about 20, or about 30, or about 40, or about 50, or about 60, or about 70, or about 80, each as measure by 100 D-antigen Units/ml.

In one aspect, the effective amount of the vaccine or antigen in the formulations of this disclosure comprises at least about 15% D-antigen recovery or an equivalent thereof. In a further aspect, the effective amount of the vaccine or antigen in the formulations of this disclosure comprises from about 15% D-antigen recovery to about 20% D-antigen recovery, or from about 20% D-antigen recovery to about 25% D-antigen recovery, or from about 25% D-antigen recovery to about 30% D-antigen recovery, or from about 30% D-antigen recovery to about 35% D-antigen recovery, or from about 35% D-antigen recovery to about 40% D-antigen recovery, or from about 40% D-antigen recovery to about 45% D-antigen recovery, or from about 45% D-antigen recovery to about 50% D-antigen recovery, or from about 50% D-antigen recovery to about 55% D-antigen recovery, or from about 55% D-antigen recovery to about 60% D-antigen recovery, or from about 60% D-antigen recovery to about 65% D-antigen recovery, or from about 65% D-antigen recovery to about 70% D-antigen recovery, or from about 70% D-antigen recovery to about 75% D-antigen recovery, or from about 75% D-antigen recovery to about 80% D-antigen recovery, or from about 80% D-antigen recovery to about 85% D-antigen recovery, or from about 85% D-antigen recovery to about 90% D-antigen recovery, or from about 90% D-antigen recovery to about 95% D-antigen recovery, or from about 95% D-antigen recovery to about 98% D-antigen recovery.

In one aspect, the effective amount of the vaccine or antigen in the formulations of this disclosure comprises at least about 15% D-antigen recovery as measured using size-exclusion high-performance liquid chromatography (SE-HPLC) or an equivalent thereof. In a further aspect, the effective amount of the vaccine or antigen as measured by using size-exclusion high-performance liquid chromatography (SE-HPLC) or an equivalent thereof to screen for the effective amount of the vaccine or antigen in the formulations of this disclosure comprises from about 15% D-antigen recovery to about 20% D-antigen recovery, or from about 20% D-antigen recovery to about 25% D-antigen recovery, or from about 25% D-antigen recovery to about 30% D-antigen recovery, or from about 30% D-antigen recovery to about 35% D-antigen recovery, or from about 35% D-antigen recovery to about 40% D-antigen recovery, or from about 40% D-antigen recovery to about 45% D-antigen recovery, or from about 45% D-antigen recovery to about 50% D-antigen recovery, or from about 50% D-antigen recovery to about 55% D-antigen recovery, or from about 55% D-antigen recovery to about 60% D-antigen recovery, or from about 60% D-antigen recovery to about 65% D-antigen recovery, or from about 65% D-antigen recovery to about 70% D-antigen recovery, or from about 70% D-antigen recovery to about 75% D-antigen recovery, or from about 75% D-antigen recovery to about 80% D-antigen recovery, or from about 80% D-antigen recovery to about 85% D-antigen recovery, or from about 85% D-antigen recovery to about 90% D-antigen recovery, or from about 90% D-antigen recovery to about 95% D-antigen recovery, or from about 95% D-antigen recovery to about 98% D-antigen recovery or an equivalent thereof.

In one aspect, the effective amount of the vaccine or antigen in the formulations of this disclosure comprises at least about 15% D-antigen recovery as measured using enzyme-linked immunosorbent assay (ELISA) or an equivalent thereof. In a further aspect, the effective amount of the vaccine or antigen as measured by using enzyme-linked immunosorbent assay (ELISA) or an equivalent thereof to screen for the effective amount of the vaccine or antigen in the formulations of this disclosure comprises from about 15% D-antigen recovery to about 20% D-antigen recovery, or from about 20% D-antigen recovery to about 25% D-antigen recovery, or from about 25% D-antigen recovery to about 30% D-antigen recovery, or from about 30% D-antigen recovery to about 35% D-antigen recovery, or from about 35% D-antigen recovery to about 40% D-antigen recovery, or from about 40% D-antigen recovery to about 45% D-antigen recovery, or from about 45% D-antigen recovery to about 50% D-antigen recovery, or from about 50% D-antigen recovery to about 55% D-antigen recovery, or from about 55% D-antigen recovery to about 60% D-antigen recovery, or from about 60% D-antigen recovery to about 65% D-antigen recovery, or from about 65% D-antigen recovery to about 70% D-antigen recovery, or from about 70% D-antigen recovery to about 75% D-antigen recovery, or from about 75% D-antigen recovery to about 80% D-antigen recovery, or from about 80% D-antigen recovery to about 85% D-antigen recovery, or from about 85% D-antigen recovery to about 90% D-antigen recovery, or from about 90% D-antigen recovery to about 95% D-antigen recovery, or from about 95% D-antigen recovery to about 98% D-antigen recovery or an equivalent thereof.

In one aspect, the formulations of this disclosure are lyophilized.

In a further aspect, wherein the lyophilized vaccine formulation of this disclosure has a moisture content of no more than about 2% water or an equivalent thereof. In one aspect, wherein the lyophilized vaccine formulation of this disclosure has a moisture content comprising from about 0.2% water to about 0.5% water, from about 0.5% water to about 0.75% water, from about 0.75% water to about 1% water, from about 1% water to about 1.25% water, from about 1.25% water to about 1.5% water, from about 1.5% water to about 1.75% water, or from about 1.75% water to about 2% water or an equivalent thereof.

The formulations can be combined into kits that in one aspect, contain instructions for use.

The formulations are useful to immunize a subject against a viral infection or viral-related disease by administering to a subject in need thereof an effective amount of the formulation. Modes of administration are known in the art and can comprise one or more doses. Subjects include animals and mammals, as well as human patients, such as infants, juveniles and adults.

### Methods For Preparing the Vaccine Formulations

Also provided herein are methods to prepare the formulations, comprising, or alternatively consisting essentially of, or yet further consisting of, admixing an effective amount of each of the components to provide a vaccine formulation comprising:
a. an effective amount of a virus or viral antigen;
b. from about 2% to about 10% of a bulking agent;
c. from about 5 mM to about 25 mM of a buffer;
d. from about 0.5% to about 1.5% of a sugar composition; and
e. from about 0.1% to about 10% or from about 0.5 mM to about 1.5 mM of a stabilizer

and adjusting the pH from about 5.5 to about 8.5.

In one aspect, the method can further comprise admixing from about 0.005% to about 1.5% of a surfactant solution.

In one aspect, the method can further comprise admixing from about 0.5 mM to about 1.5 mM of a metal ion.

In one aspect, the bulking agent to be admixed to a final concentration is from about 2% to about 10% of the bulking or an equivalent thereof. In one aspect, the bulking agent is Mannitol or Glycine, or an equivalent of each thereof, and an equivalent of Mannitol or Glycine intends other known bulking agents that provide the same or similar effectiveness and stability of the above-noted formulation. In another aspect the bulking agent such as Mannitol or Glycine, or an equivalent of each thereof, is provided in an amount from about 3% to about 9%, or alternatively from about 4% to about 8%, or alternatively from about 3% to about 8%, or alternatively from about 3% to about 7%, or alternatively from about 4% to about 7%, or alternatively from about 4% to about 6%, or alternatively about 5%. In one aspect, the bulking agent is about 5% of Mannitol or Glycine, or an equivalent of each thereof. In one aspect, the bulking agent is about 2.5% of Mannitol or Glycine, or an equivalent of each thereof.

In another aspect, the buffer to be admixed in the formulation to a final concentration is from about 5 mM to about 25 mM, or alternatively from about 5 mM to about 20 mM, or from about 10 mM to about 20 mM, or from about 15 mM to about 25 mM, or from about 15 mM to about 20 mM, or from about 5 mM to about 10 mM, or from 8 mM to about 12 mM, or about 10 mM. In one aspect the buffer comprises or is histidine or Tris-HCl, or an equivalent of each thereof. An equivalent of histidine or Tris-HCl intends other known buffering agents that provide the same or similar effectiveness and stability of the above-noted formulation comprising histidine or Tris-HCl. In one aspect, the buffer is about 10 mM of histidine or Tris-HCl, or an equivalent of each thereof.

In another aspect, the sugar to be admixed comprises a sugar to a final concentration at about 0.5% to about 1.5%, or alternatively from about 0.7% to about 1.5%, or alternatively from about 0.9% to about 1.5%, or alternatively from about 1.0% to about 1.5%, or alternatively from about 0.5% to about 1.2%, or alternatively from about 0.7% to about 1.2%, or alternatively from about 0.9% to about 11%, or alternatively about 1.0%. In one aspect the sugar is sucrose or sorbitol, or an equivalent of each thereof. An equivalent of sucrose or sorbitol intends other known sugars provided in vaccine formulations that provide the same or similar effectiveness and stability of the above-noted formulation.

In a further aspect, the surfactant to be admixed in the formulation to a final concentration comprises from about 0.005% to about 1.5% of a surfactant solution. Alternatively, the vaccine formulation comprises from about 0.01% to about 0.5%, or about 0.01% to about 0.5%, or about 0.01% to about 0.3%, or about 0.01% to about 1.0%, or about 0.5% to about 1.5%, or about 0.001% to about 0.007%, or about 0.001% to about 0.003%, or about 0.001% to about 0.05%, or about 0.1%, of a surfactant solution. In one aspect, the surfactant solution comprises of polysorbate 20, polysorbate 80, or poloxamer 188 (pluronic F68). In a further aspect, the surfactant comprises 0.01% of polysorbate 20.

In a yet further aspect, the metal ion to be admixed to a final concentration comprises 0.5 mM to about 1.5 mM of a metal ion, or from about 0.5 mM to about 1.3 mM, or from about 0.7 mM to about 1.5 mM, or from about 0.9 mM to about 1.5 mM, or from about 0.7 mM to about 1.2 mM, or from about 0.8 mM to about 11 mM, or about 0.9 mM to about 1.1 mM, or about 1 mM of the metal ion. In one aspect the metal ion comprises from about 0.5 mM to about 1.5 mM of MgSO₄ or MgCl₂, or an equivalent or each thereof. In a further aspect, the metal ion comprises about 1 mM of MgSO₄, or an equivalent thereof. An equivalent of MgSO₄ or MgCl₂ intends other known metal ions used in vaccine formulations that provide the same or similar effectiveness and stability of the above-noted formulation.

In another aspect, the stabilizer to be admixed to a final concentration comprises from about 0.1% to 10% of a stabilizer, or from about 0.5% to about 8%, or from about 0.5% to about 8%, or from about 0.5% to about 8%, or from about 0.5% to about 8%, or from about 0.5% to about 5%, or from about 0.5% to about 3%, or about 1% of the stabilizer. In one aspect the stabilizer comprises polyvinylpyrrolidone or polyethylene glycol, or an equivalent thereof. An equivalent of polyvinylpyrrolidone or polyethylene glycol intends other known vaccine stabilizers used in vaccine formulations that provide the same or similar effectiveness and stability of the above-noted formulation. In a further aspect, the stabilizer from about 1% of polyvinylpyrrolidone or an equivalent thereof. In another aspect, the vaccine formulation comprises from about 0.5 mM to about 1.5 mM of a stabilizer. In a further aspect, the stabilizer comprises from about 0.5 mM to about 1.5 of MgSO₄, or alternatively about 1 mM of MgSO₄ or an equivalent thereof. An equivalent of MgSO₄ intends other known vaccine stabilizers used in vaccine formulations that provide the same or similar effectiveness and stability of the above-noted formulation.

The pH of the formulation should be from about 5.5 to about 8.5, or from about 6.1 to about 7.5, or from about 6.2 to about 7.5, or from about 6.3 to about 7.5, or from about 6.4 to about 7.5, or from about 6.4 to about 7.5, or from about 6.5 to about 7.5, or from about 6.6 to about 7.5, or from about 6.7 to about 7.5, or from about 6.8 to about 7.5 or from about 6.9 to about 7.5, or from about 6.2 to about 7.4, or from about 6.3 to about 7.4, or from about 6.4 to about 7.3, or from about 6.4 to about 7.2, or from about 6.5 to about 7.2, or from about 6.8 to about 7.2, or from about 6.6 to about 7.1, or from about 6.9 to about 7.4, or from about 6.9 to about 7.3, or from about 6.9 to about 7.2, or from about 6.9 to about 7.2, or about 7.0.

In one aspect, the above noted formulations are provided without a virus or viral antigen. However when they do comprise the virus or viral antigen, the formulations are suitable for use in formulation of other known vaccine components, e.g., DNA or RNA-viral vaccines, and for the prevention of disease, examples of which can be found on the World Health Organization website. Non-limiting examples of such include, diphtheria, Hepatitis B, Heamohilus influenza type B, Human papillomavirus, influenza, measles, mumps, pertussis, Rubella, Pneumococcal diseases, poliomyelitis (polio), Rotavirus, Tuberculosis, Tetnus, Varicella, Cholera, Hepatitis A, Hepatitis B, Japanese encephalitis, Meningococcal disease, Rabies, Tick-born encephalitis, Typhoid fever, Yellow fever, Dengue, Enterovirus 71, HIV-1, Leishmaniasis Disease, Malaria, Shigella and Ebola.

In one particular aspect, the virus comprises formaldehyde-inactivated polio virus. In another aspect, the virus or viral antigen comprises D-antigen units of a heat-inactivated polio vaccine.

In one aspect, the effective amount of the formaldehyde-inactivated polio virus to a final amount is from about 0.3×10⁷ to about 10.0×10⁷ pfu/ml, or from about 0.5×10⁷ to about 10.0×10⁷ pfu/ml, or from about 0.5×10⁷ to about 8.0×10⁷ pfu/ml, or from about 0.5×10⁷ to about 6.0×10⁷ pfu/ml, or from about 0.4×10⁷ to about 10.0×10⁷ pfu/ml, or from about 0.5×10⁷ to about 10.0×10⁷ pfu/ml, or from about 0.5×10⁷ to about 9.0×10⁷ pfu/ml, or from about 0.8×10⁷ to about 10.0×10⁷ pfu/ml, or from about 0.5×10⁷ to about 9.0×10⁷ pfu/ml, or about 0.6, or about 0.7, or about 0.8, or about 0.9, or about 1.0, or about 1.5, or about 2.0, or about 3.5, or about 4.0, or about 4.5, or about 5.0, or about 5.5, or about 6.0, each as X 10⁷ pfu/ml. In one aspect, the effective amount of the heat-inactivated polio virus is from about 0.6×10⁷ to about 7.0×10⁷ pfu/ml.

In a further aspect, when the viral antigen to a final amount comprises D-antigen units of a heat-inactivated polio vaccine, the effective amount of the vaccine or antigen comprises from about 20 D-antigen Units/ml to about 100 D-antigen Units/ml, or from about 30 D-antigen Units/ml to about 90 D-antigen Units/ml, 40 D-antigen Units/ml to about 100 D-antigen Units/ml, or from about 50 D-antigen Units/ml to about 100 D-antigen Units/ml, or from about 60 D-antigen Units/ml to about 100 D-antigen Units/ml, or from about 70 D-antigen Units/ml to about 100 D-antigen Units/ml, or from about 70 D-antigen Units/ml to about 90 D-antigen Units/ml, or from about 75 D-antigen Units/ml to about 85 D-antigen Units/ml, or about 20, or about 30, or about 40, or about 50, or about 60, or about 70, or about 80, each as measure by 100 D-antigen Units/ml.

In one aspect, the effective amount of the vaccine or antigen in the formulations of this disclosure comprises at least about 15% D-antigen recovery or an equivalent thereof. In a further aspect, the effective amount of the vaccine or antigen in the formulations of this disclosure comprises from about 15% D-antigen recovery to about 20% D-antigen recovery, or from about 20% D-antigen recovery to about 25% D-antigen recovery, or from about 25% D-antigen recovery to about 30% D-antigen recovery, or from about 30% D-antigen recovery to about 35% D-antigen recovery, or from about 35% D-antigen recovery to about 40% D-antigen recovery, or from about 40% D-antigen recovery to about 45% D-antigen recovery, or from about 45% D-antigen recovery to about 50% D-antigen recovery, or from about 50% D-antigen recovery to about 55% D-antigen recovery, or from about 55% D-antigen recovery to about 60% D-antigen recovery, or from about 60% D-antigen recovery to about 65% D-antigen recovery, or from about 65% D-antigen recovery to about 70% D-antigen recovery, or from about 70% D-antigen recovery to about 75% D-antigen recovery, or from about 75% D-antigen recovery to about 80% D-antigen recovery, or from about 80% D-antigen recovery to about 85% D-antigen recovery, or from about 85% D-antigen recovery to about 90% D-antigen recovery, or from about 90% D-antigen recovery to about 95% D-antigen recovery, or from about 95% D-antigen recovery to about 98% D-antigen recovery.

In one aspect, the method can further comprise using size-exclusion high-performance liquid chromatography (SE-HPLC) or an equivalent thereof to screen for the effective amount of the vaccine or antigen comprising of at least about 15% D-antigen recovery or an equivalent thereof following lyophilization. In a further aspect, the effective amount of the vaccine or antigen as measured by using size-exclusion high-performance liquid chromatography (SE-HPLC) or an equivalent thereof to screen for the effective amount of the vaccine or antigen in the formulations of this disclosure comprises from about 15% D-antigen recovery to about 20% D-antigen recovery, or from about 20% D-antigen recovery to about 25% D-antigen recovery, or from about 25% D-antigen recovery to about 30% D-antigen recovery, or from about 30% D-antigen recovery to about 35% D-antigen recovery, or from about 35% D-antigen recovery to about 40% D-antigen recovery, or from about 40% D-antigen recovery to about 45% D-antigen recovery, or from about 45% D-antigen recovery to about 50% D-antigen recovery, or from about 50% D-antigen recovery to about 55% D-antigen recovery, or from about 55% D-antigen recovery to about 60% D-antigen recovery, or from about 60% D-antigen recovery to about 65% D-antigen recovery, or from about 65% D-antigen recovery to about 70% D-antigen recovery, or from about 70% D-antigen recovery to about 75% D-antigen recovery, or from about 75% D-antigen recovery to about 80% D-antigen recovery, or from about 80% D-antigen recovery to about 85% D-antigen recovery, or from about 85% D-antigen recovery to about 90% D-antigen recovery, or from about 90% D-antigen recovery to about 95% D-antigen recovery, or from about 95% D-antigen recovery to about 98% D-antigen recovery, or an equivalent thereof.

In a further aspect, the method can further comprise using enzyme-linked immunosorbent assay (ELISA) to screen for the effective amount of the vaccine or antigen comprising of at least about 15% D-antigen recovery or an equivalent thereof following lyophilization. In a further aspect, the effective amount of the vaccine or antigen as measured by using enzyme-linked immunosorbent assay (ELISA) or an equivalent thereof to screen for the effective amount of the vaccine or antigen in the formulations of this disclosure comprises from about 15% D-antigen recovery to about 20% D-antigen recovery, or from about 20% D-antigen recovery to about 25% D-antigen recovery, or from about 25% D-antigen recovery to about 30% D-antigen recovery, or from about 30% D-antigen recovery to about 35% D-antigen recovery, or from about 35% D-antigen recovery to about 40% D-antigen recovery, or from about 40% D-antigen recovery to about 45% D-antigen recovery, or from about 45% D-antigen recovery to about 50% D-antigen recovery, or from about 50% D-antigen recovery to about 55% D-antigen recovery, or from about 55% D-antigen recovery to about 60% D-antigen recovery, or from about 60% D-antigen recovery to about 65% D-antigen recovery, or from about 65% D-antigen recovery to about 70% D-antigen recovery, or from about 70% D-antigen recovery to about 75% D-antigen recovery, or from about 75% D-antigen recovery to about 80% D-antigen recovery, or from about 80% D-antigen recovery to about 85% D-antigen recovery, or from about 85% D-antigen recovery to about 90% D-antigen recovery, or from about 90% D-antigen recovery to about 95% D-antigen recovery, or from about 95% D-antigen recovery to about 98% D-antigen or an equivalent thereof.

In one aspect, the formulations of this disclosure are lyophilized.

In a further aspect, wherein the lyophilized vaccine formulation of this disclosure has a moisture content of no more than about 2% water or an equivalent thereof. In one aspect, wherein the lyophilized vaccine formulation of this disclosure has a moisture content comprising from about 0.2% water to about 0.5% water, from about 0.5% water to about 0.75% water, from about 0.75% water to about 1% water, from about 1% water to about 1.25% water, from about 1.25% water to about 1.5% water, from about 1.5% water to about 1.75% water, or from about 1.75% water to about 2% water or an equivalent thereof.

In one aspect, the methods further comprise admixing a preservative, lyoprotectant and/or a cryoprotectant. In a yet further aspect, the method comprises lyophilizing the formulations.

Formulations prepared or obtained by these methods are further provided herein.

### Experiment No. 1

### Materials and Methods

### Tissue Culture

Vero E6 cells and HeLa cells were purchased from ATCC and maintained in Dulbecco's modified Eagle's medium (DMEM; Gibco) containing 10% FBS and 1% Penicillin-streptomycin. Transient transfections were performed with PEI according to the manufacturer's instructions [24]. Large scale culturing of vero cells were done in a 3 L spinner flask (Corning) at 40 rpm after attaching vero cells to micro carrier beads (Sigma). Media was changed every two days by removing half of the media and reintroducing fresh media.

### Virus Culture

Hela cells were co-transfected with pREV plasmid encoding T7 RNA polymerase and Sabin RNA encoding plasmid for 96 hrs. Once 100% CPE was confirmed after 96 hrs, remaining cells were scraped with a cell lifter and the supernatant was collected. The supernatant was freeze and thawed three times and passed through a 0.2 micrometer PES filter. The clarified supernatant was loaded at MOI 10 into a dish of Vero cells and the cells and supernatant were collected after overnight incubation. After the supernatant was clarified, the virus suspension was loaded at MOI 30 into a suspension of Vero cells in a spinner flask. After overnight infection, the supernatant was collected and freeze and thawed three times.

### Viral Purification

Viral suspension was first clarified by centrifugation at 4000 rpm for 30 minutes and passed through a 0.2 micrometer PES filter. The clarified suspension was concentrated in a labscale tangential flow filtration system (Millipore) using a biomax 100 kDa cartridge (Millipore) at 20 psi input and 10 psi back pressure. Viral concentrate was loaded into a Sephacryl HR-200 column (GE) and run through 20 mM sodium phosphate buffer pH 7.0 at 0.5 mL/min on a Biorad Duoflow Chromatography system. The first UV 280 nm absorption peak was collected and loaded into a DEAE Fast Flow Column (GE) using 20 mM sodium phosphate buffer at pH 7.0, and the flow through was collected. The purity of virus was confirmed by loading the SDS-treated virion on PAGE-gel, followed by silver staining.

### Viral Inactivation

Methanol-free formaldehyde was added into the purified poliovirus suspension at a final concentration of 0.025%. 10× M199 media was also added to the suspension. The suspension was then incubated at 37 degrees for 14 days. The suspension was passed through a 0.2 micrometer PES filter on day 7 to remove any aggregates forming. On day 14, sodium bisulfite was added to neutralize the formaldehyde. The suspension was then dialyzed using a 10 kDa slide-a-lyzer cassette (Thermo) back into 20 mM sodium phosphate buffer and 25 mM NaCl using manufacturer's instructions.

### D-Antigen Unit ELISA

ELISA plates were coated with bovine serum anti-poliovirus (NIBSC) antibody at a concentration of 1:100. The sIPV was loaded at two fold dilutions and a WHO standard IPV (Nibsc) was used to produce a standard curve. Mouse monoclonal antibody against type 1 poliovirus (abcam) diluted at 1:1000 was added for detection, and secondary anti-mouse hrp antibody (Cell Signaling) diluted at 1:1000 was loaded. TMB solution (eBioscience) was used for quantification on a FilterMax F5 micro plate reader (Molecular Devices) after the reaction was stopped using hydro sulfuric acid.

### Viral Titration

Vero cells were cultured to confluence in 6 well plates. After washing with PBS, 500 microliters of viral dilutions from added to each well. Following 90 minute incubation of the 10 fold viral dilutions in 37 degrees, the viral dilutions were aspirated and a 0.75% avicel in DMEM overlay was placed on the cells. 6 days later, the overlay was removed, 4% formaldehyde was used to fix the cells, and they were stained with crystal violet.

### SE-HPLC

SE-HPLC was performed with an Agilent 1100 series instrument (Santa Clara, Calif.) equipped with quaternary pump, degasser, temperature controlled autosampler, a UV/Vis DAD and an Agilent 1200 series fluorescence detector (FLD). TSKgel G6000PW_{XL} (7.8 mm×30 cm) or TSKgel G3000SW_{XL} (7.8 mm×30 cm) column purchased from Tosoh Bioscience (King of Prussia, Pa.) was used. At a flow rate of 0.8 mL/min, 100 μL of sample was injected per analysis. The mobile phase contained 50 mM Sodium Phosphate, 140 mM NaCl at pH 6.7. The FLD was used as the primary detector and set to acquire data at an excitation of 280 nm and an emission of 336 nm. For Polio vaccine peak identification, the elution volume and the corresponding retention time of the FLD signal was calculated based on standard calibration curve. To calculate Polio vaccine recovery, the area under the curve of formulations before lyophilization was compared to the area under the curve of formulations after lyophilization.

### DLSA

Dynamic Light Scattering measurements to obtain the mean radius of IPV were performed with Wyatt Technology's (Santa Barbara, Calif.) DynaPro Plate Reader. IPV samples were prepared by diluting the vaccine 1:1 with phosphate buffer and filtered through 0.2 μm for DLS analysis. The samples were analyzed at 25 μL volume in triplicate and the mean particle radius for the vaccine calculated via the Dynamics software (version 7.1.7).

### Lyophilized Cake Moisture Determination

The water (or moisture) content in solid lyophilized formulation was determined by coulometric titration using a Mettler Karl Fischer (KF) titrator (Coulometer C20; Mettler Toledo, Ohio) and an oven. Briefly, each lyophilized sample was brought to room temperature before caps were removed for analysis and subsequently heated in an oven. The moisture released from heating was carried from the oven to the KF titration cell which contained KF reagent for the reaction. Moisture in the titration cell was continuously titrated until an endpoint was reached. Each sample was measured in duplicate. Once an analysis was complete, results were generated automatically.

### Formulation Matrix

All formulations were prepared at twice the concentration to allow for final 1:1 dilution by volume with the IPV sample. Each stock formulation was prepared by dissolving the stabilizers and bulking agents into 10 mM Histidine buffer containing either polysorbate 20 or Poloxamer 188. The adjustment of pH was performed with HCl prior to sterile filtration using 0.2 μm PES membrane. The final IPV-formulation solutions were prepared by mixing equal volumes of IPV sample and stock formulation to generate the formulation matrix for testing. Each mixture was filled into sterilized 2 cc glass vials (West Pharmaceutical, Exton, Pa.) in two (2) replicates and half-capped with sterilized Diakyo 13 mm serum Flurotec stoppers (West Pharmaceutical, Exton, Pa.). All sample preparation and the filling process were performed under aseptic conditions in a Class II biological safety cabinet.

Lyophilization Process Design of sIPV

For this study, all formulations were lyophilized using a conservative cycle designed to generate elegant lyophilized cakes with acceptable moisture content without compromising the vaccine product quality and integrity. The half-capped vials were loaded into a VirTis Genesis 25EL pilot lyophilizer (SP Scientific, Gardiner, N.Y.) at a shelf temperature of 5° C. Following loading, the vials were slowly frozen to −50° C., held at the same temperature for 2 hours and subsequently warmed to −15° C. at 0.5° C./min ramp rate. The vials were held at −15° C. for 2 hours prior to initiating primary drying step. The primary drying was performed at −15° C. shelf temperature with 100 mTorr chamber pressure for 10 hours. The secondary drying step was designed to remove residual water that did not sublimate during the primary drying step; thus, the shelf temperature was increased to 25° C. and held for 3 hours. After the completion of the lyophilization cycle, the vials were stoppered in partial vacuum, labeled and stored at 2-8° C. before analysis.

### Animal Care

Transgenic Polio Virus Receptor Mice were a gift from Dr. Raul Andino (Stanford University) and were maintained in USC mice facility according to the university's regulation for animal care and handling (IACUC).

### Poliovirus Challenge

6 week old cPVR mice (n=8) were vaccinated with ½ of a human dose of sIPV intraperitoneally and boosted with the same dose 14 days later. 14 days following the booster, the mice were injected with wild type 1 Mahoney poliovirus at a dose of 50 times paralytic dose 50 (PD50). The mice were monitored for two weeks using a blinded paralysis scoring system outlined by WHO for mouse neurovirulence test for oral polio vaccine. If both legs were dragged during ambulatory motions, legs hanged when climbing across a rail, and unable to grip the rail, they were scored a paralyzed. If the mice maintained partial ability to move limb forward, legs hanged when climbing across a rail but recovered, and unable to grip the rail, the mice were scored a paresis with a paralyzed score if showing signs of paresis for two consecutive days. Mice were euthanized after showing scoring as paralyzed for a humane end point.

### Neutralization Assay

Neutralization assay followed WHO standardized protocol for neutralization assay in rats [23]. Blood was collected from retro-orbital draw on the 21^{st} day following a day 1 vaccination and a day 14 booster. The blood was allowed to clot at 4 degree for two days, and then centrifuged at 1000 rpm for 30 min at 4 degrees in order to collect the serum. The serum was diluted by ¼ and loaded into two columns of the first row in 96 well plates. A two fold dilution was made to the serum on each row of the 96 well plate up to 1/512, and the final row was used as a serum control with the original ¼ dilution. 100TCID of the virus in equal volume to serum was added to each well and incubated for 3 hrs in 37 degrees. The plate was then further incubated at 4 degree overnight. After overnight incubation, 1×10\hat{ }5 cells were added to each well and cultured for 5 days. On the 5^{th} day, cells were fixed with 4% formaldehyde and stained with crystal violet.

### Results

Development of High Throughout HPLC Analysis of sIPV

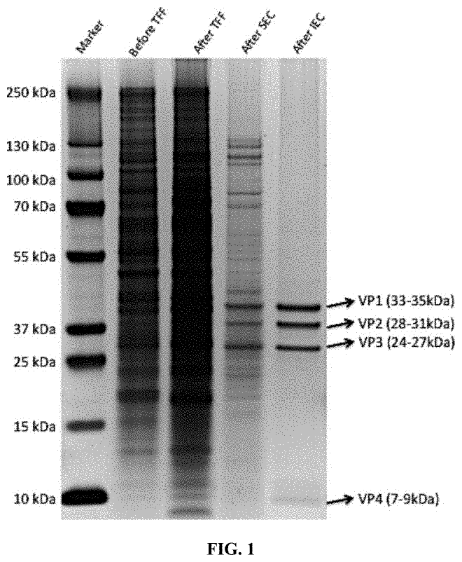

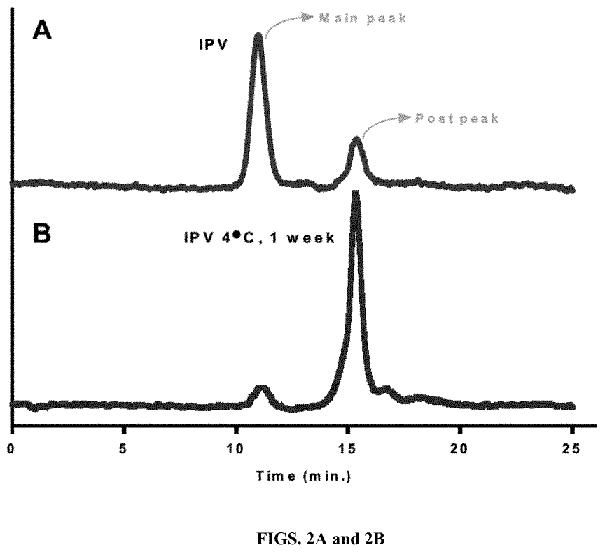

Poliovirus vaccines commonly contain both a D-antigen form and a C-antigen form, where the C-antigen form does not contain the poliovirus viral RNA and cannot induce protection against poliovirus infection [13]. Additionally, the C-antigen is known as the heated antigen because the D-antigen can convert to the C-antigen when exposed to high temperatures. Therefore, to optimize all conditions for a lyophilized IPV formulation, a high throughput sIPV stability analysis needed to be established for D-antigen quantification instead of the time consuming D-antigen ELISA. First, sIPV stocks with concentrated D-antigen units were produced in house using scaled down methods of commercial IPV production [14, 15]. To analyze the D-antigen stability, Size Exclusion High Performance Liquid Chromatography (SE-HPLC) appeared a potential candidate to confirm both the stability of the D-antigen and sample purity. 336 nm emission detection from 280 nm excitation of sIPV resulted in consistent baseline with little noise, and the chromatogram consistently showed two distinct peaks (FIG. 2A and Table 1). The earlier peak from the left will be called the main peak, and the later peak on the right will be called the post peak from this point. A silver stained protein gel revealed no protein contaminants beyond the expected VP1, 2, 3, and 4 of the poliovirus (FIG. 1) [16]. Thus, the heavier, RNA filled D-antigen particle likely presented the main peak and the C-antigen in the post peak.

Dynamic light scatter analysis, commonly used to measure various liquid suspended particle radii, revealed that the main peak contained a mono-disperse particle of 14.6 nm (Table 2), which matches closely with the expected poliovirus radius of ˜15 nm [16]. Elutate from the main peak and post peak were both collected and D-antigen content in each peak was measured by ELISA (FIG. 2A). The main peak showed reactivity to the type 1 poliovirus antibody, while the post peak did not bind. Finally, to confirm a relationship between the main and post peak, the elution from the main peak was collected and passed through the SE-HPLC again. This revealed a significant increase in the post peak area and a decrease in the main peak area, but the combined total area of the two peaks equaled that of the original main peak. With confidence that the main peak from the SE-HPLC can help identify D-antigen content in the sIPV sample, Applicants ran a preliminary stability test by storing the sIPV in 4° C. for one weak and running the sample back into the SE-HPLC. Although to a much lower degree, the main peak decreased in peak area and the post peak increased compared to the sample run in the week prior, but the combined area remained constant between the first and second run (FIG. 2B and Table 1). Therefore, the SE-HPLC was chosen as a reliable source for measuring the stability of D-antigen content by monitoring the fluctuations in the total peak area of the main and post peaks.

### Surfactant Based Formulations Yield High Recovery After Lyophilization

To approach possible formulations for sIPV temperature stabilization a surfactant's effect on the vaccine was assessed through agitation studies. The ability to add surfactants to the formulation helps not only for the stability of the vaccine, but protects the vaccine from events that can possibly damage the D-antigen content such as antigens sticking to needles, sticking to glass vials, or reducing vaccine interaction with formulation components. Polysorbate 20, polysorbate 80, and pluronic F68 were added to the vaccine to achieve concentrations of 0.01%, 0.01%, and 0.1% respectively. 4 sIPV vials, each containing a different surfactant and one containing no surfactant, were vigorously agitated at 1000 rpm for 4 hrs. 4 untouched vials with the same formulation were left in ambient temperature to compare the loss in D-antigen content. sIPV formulation without the surfactant lost a significant amount of D-antigen content, while surfactant supplemented vaccine vials remained highly durable against the agitation. Agitation studies showed a clear benefit in adding surfactants to sIPV lyophilization stabilization formulas, especially polysorbate 20 and pluronic f68.

After the confirmation that surfactants addressed a vital concern for the lyophilization process, various bulking agents, sugars, and buffers needed analysis to reach a final formula. Table 3 lists the key candidates screened for the temperature stabilization along with each formulation's respective recovery of D-antigen units after lyophilization. MgSO₄ was immediately confirmed for the formulation since it is a common stabilizer for lyophilization and past literature supports the need for MgSO₄ in IPV stabilization [3]. pH marked a valuable starting point for the stabilization formula as the pH would define the types of buffers to use. Based on SE-HPLC recovery of the main peak, a pH of 6 consistently showed low recovery after lyophilization. Thereafter, formulations were constricted to pH 7. Mannitol as the bulking agent and histidine as the buffer to maintain a pH 7 provided the most consistent D-antigen unit recoveries. It appeared that either sorbitol or sucrose could be used as the sugar to maintain stability through the lyophilization cycle and produce elegant cake products, but the key component differentiating D-antigen recovery efficiency was the surfactant concentration. Minimal changes in concentration of surfactants resulted in differences as large as 20% in recovery. From the table of candidates, the formulation code F4, F8, and F9 gave the highest recovery with formulation F4 showing ELISA analyzed D-antigen unit recovery at 95% recovery. Although F5 showed a similar percent recovery as these three formulations, the formulation was avoided due to the high surfactant concentration that could cause complication later in the study. To ensure that formulations F4, F8, and F9 are clinically applicable formulations, the moisture content was analyzed and resulted in values 0.77%, 0.93%, and 1.30% respectively (Table 4).

sIPV is Thermostabilized by Lyophilization

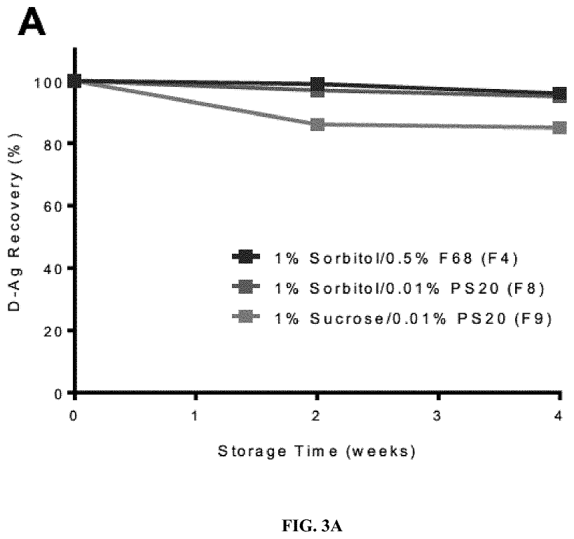

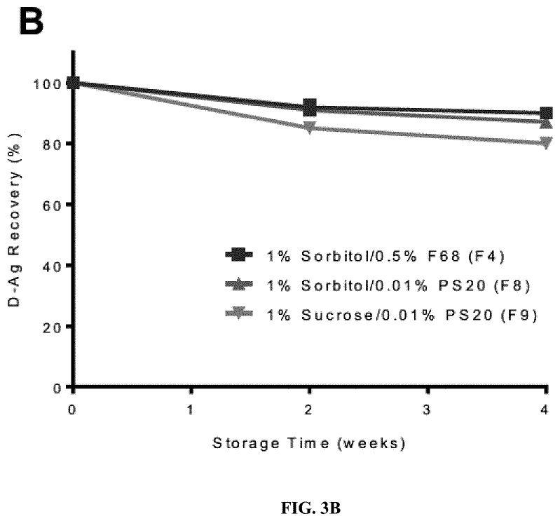

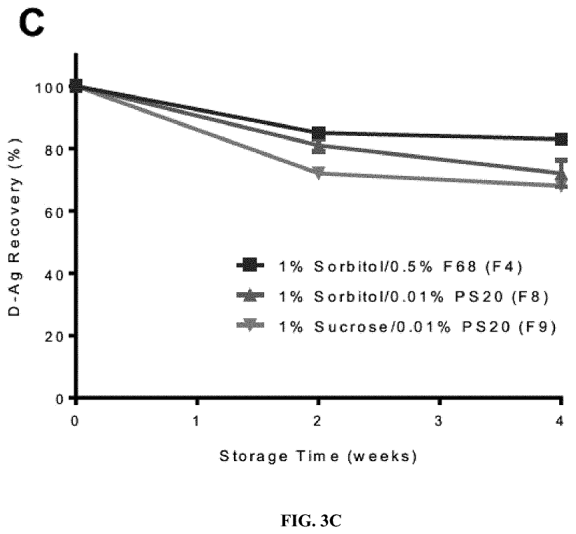

Utilizing formulations that maintain high recovery of D-antigen content after lyophilization, one month storage studies were performed to test the formulations ability to stabilize the D-antigen content at various temperatures. The pre-lyophilized sIPV and the three lyophilized sIPV candidates were incubated in 4° C., 25° C., and 40° C. At 4° C. all but one formulation showed consistent D-antigen recovery from week to week, including the pre-lyophilized sIPV (FIG. 3A). Formulation F9 dropped significantly in the first week, but plateaued in the following weeks. Meanwhile, the 25° C. incubation induced a consistent degradation in all formulations, and the pre-lyophilized sIPV degraded at a much faster rate than formulations F4 and F8 (FIG. 3B). Although formulation F9 showed worse stability, again, after 25° C. incubation, formulation F9 began to show signs of plateauing between week 3 and 4 as opposed to the consistent decrease in D-antigen units in the pre-lyophilized sIPV. After 4 weeks, the liquid pre-lyophilized sIPV maintained 15% less D-antigen content than the F4 lyophilized formulation. At 40° C., the effect of lyophilizing the sIPV revealed a clear advantage for the lyophilization of sIPV. Pre-lyophilization sIPV quickly degraded in D-antigen content over the course of a month, while all formulations, including the 4° C. and 25° C. poor performing F9 formulation, maintained at least 70% recovery over a one month period (FIG. 3C). Moreover, producing the best recoveries in both 4° C. and 25° C. as well, the F4 formulation successfully stabilized the D-antigen content to preserve 83% recovery in D-antigen content after one month.

Heat Treated Lyophilized IPV Provides Transgenic Mice with Protection Against Wild Poliovirus Challenge

It is known that the IPVs and sIPVs can induce immunogenicity against polio, but whether the lyophilized formulations can still maintain the same level of immunity is not verified in vivo [17]. Following the confirmation that formulation F4 produces the best candidate for sIPV temperature stabilization, formulation F4 sIPV was prepared for vaccination in cPVR transgenic mice, which were necessary because mice do not naturally express the poliovirus receptor for viral entry [18]. To explore the ability for transgenic poliovirus receptor mice to build immunity against poliovirus from a reconstituted lyophilized sIPV, six groups of eight mice were vaccinated with following vaccines: PBS, commercial IPV, sIPV incubated in 4° C. for 4 weeks, sIPV incubated in 37° C. for 4 weeks, lyophilized sIPV incubated in 4° C. for 4 weeks, and lyophilized sIPV incubated in 37° C. for 4 weeks.

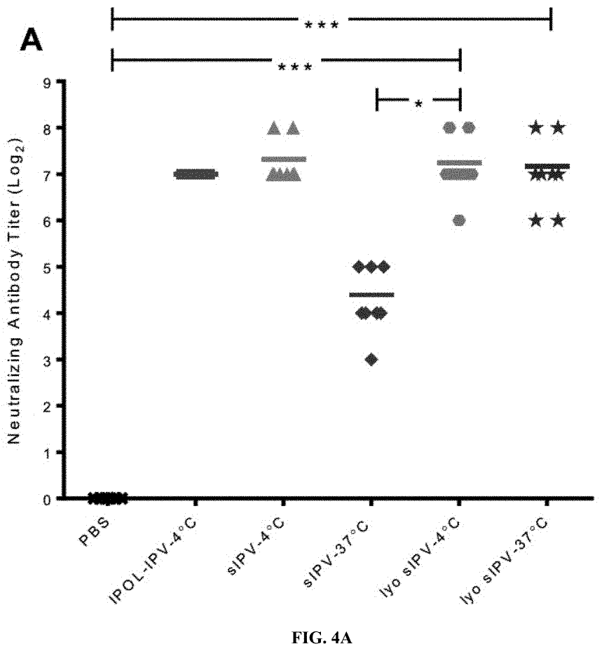

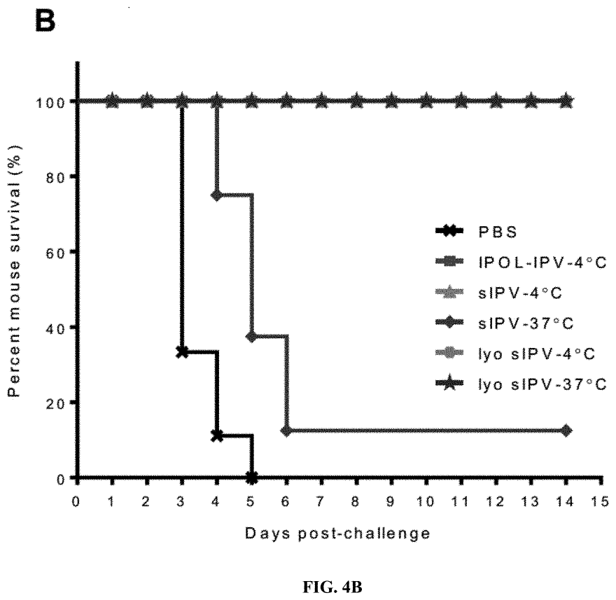

The mice were vaccinated, subsequently boosted two weeks later, and finally challenged with wild type 1 Mahoney strain virus two weeks after boosting [19, 20, 21]. The mice were observed for two weeks for signs of paralysis using a blinded scoring method outlined in the WHO standard operating procedure for OPV neurovirulence testing [22]. Additionally, adhering to WHO standardized in vivo potency testing of IPV in rats, serum was collected from mice one week after the booster for a neutralization assay [23]. A serum neutralization assay against Sabin type 1 poliovirus from serum samples taken before the vaccinations confirmed anti-poliovirus antibodies were not present in any mice. The serum samples collected on the 21^{st} day incubated with 100TCID50 showed that mice produced type 1 poliovirus neutralizing antibodies at similar levels whether or not the sIPV was lyophilized (FIG. 4A). Furthermore, the neutralizing antibody titer was significantly lower in the sIPV incubated in 37° C. for 4 weeks, but the serum antibody against type 1 poliovirus was consistent with commercial vaccine when vaccinated with the lyophilized sIPV incubated in 37° C.

In the challenge model, the transgenic poliovirus receptor mice could survive wild type 1 Mahoney strain poliovirus challenge if vaccinated by lyophilized sIPV even if the lyophilized sIPV was incubated at 37° C. However, Applicant saw that the PBS and pre-lyophilized sIPV incubated at 37° C. were unable to protect the mice from paralysis once infected by polio. All mice vaccinated with commercial vaccine, sIPV incubated in 4° C., and lyophilized sIPV incubated in 4° C. and 37° C. were immune to paralysis from poliovirus (FIG. 4B).

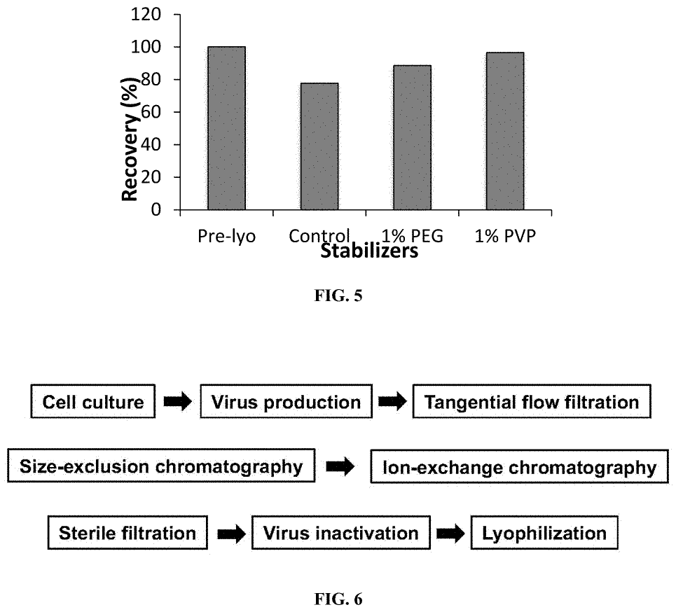

The benefit of additional stabilizer was evaluated by determining the recovery of virus after lyophilization (FIG. 5). All tested formulations contain the composition of the control formulation at 5% mannitol, 1% sucrose, 1 mM MgSO4, 0.01% polysorbate 20, in 10 mM histidine at pH 7.0. Pre-lyo sample is the sample without the lyophilization. Addition of 1% PEG (polyethylene glycol) or 1% PVP (Polyvinylpyrrolidone or Povidone) improved the recovery from 80% of the control to 95-98%, respectively.

### Discussion

This study establishes a lyophilized formulation for type 1 sIPV that can stabilize sIPV against D-antigen degradation at temperatures up to 40° C. for at least one month. By analyzing the various surfactants' and their concentrations' effect on overall stability and lyophilization recovery, Applicant now see a key component to ensure the stability of sIPV. Three formulations provided high D-antigen recovery from the lyophilization process, while maintaining moisture content less than 2%. Applicants showed that the F4 formulation in particular resulted in higher D-antigen recovery after one month compared to previous attempts to lyophilize IPV, which resulted in lower recovery from lyophilization for samples more stable at 37° C. In addition, approaching the end game of polio eradication means the shift from IPV to sIPV to avoid manufacturing borne polio infections. Thus, this novel study that Applicants' lyophilized type 1 sIPV formulations can maintain D-antigenicity equal to or higher than IPV is crucial evidence to accelerate the shift away from conventional IPV. The in vivo study revealed that the lyophilized sIPV formulations can still induce anti-poliovirus antibodies in transgenic polio virus receptor mice, and the induced antibodies can protect the mice from wild poliovirus infection.

Furthermore, the novel approach for high throughput evaluation of D-antigen content provides the opportunity to accelerate the rate Applicant can optimize each component of the formulation. With the cost and time constraints from the currently used D-antigen content, the SE-HPLC analysis of polio vaccine could help to improve a wide array of poliovirus research beyond formulation testing. Applicants' study never standardized the SE-HPLC luminescence output with D-antigen units, but this would be an important verification if SE-HPLC were able to replace D-antigen ELISAs for in vitro potency assays.

Although the lyophilization formulations makes a big difference when protecting sIPV from 40° C., the D-antigen content continues to decrease at 25° C. In order to safety remove the cold chain requirement for sIPV, maintaining a consistent D-antigen content for the duration of the month will be paramount to poliovirus eradication. Therefore, further optimization of the formulation will be necessary to achieve a truly temperature stable polio vaccine. Optimizing the temperature stability will start with ensuring a higher recovery of D-antigen units after lyophilization, until 100% recovery can be achieved. Without being bound by theory, Applicants believe that optimization will assist temperature stability, but also mitigate complications in the future if sIPV shortages begin to occur due to ever decreasing demand of global IPV production.

### Experiment No. 2

This example is an extension of the study described in Experiment No. 1.

Attempts to lyophilize IPV have resulted in low recovery following lyophilization and poor stability at ambient temperatures [17, 19-21]. While sufficient optimization of a lyophilized vaccine can substantially improve thermostability [21], it can become a cumbersome process without an efficient and effective in vitro method to evaluate vaccine potency. The potency of IPV by the in vitro assay is expressed in arbitrarily defined D-antigen units (D-AgU). The D-AgU was established in the early 1960s [22] following characterization of purified virus preparations by sucrose gradient centrifugation where two bands were identified. One, the D fraction (D-antigen), was associated with infectious virus with intact structure as revealed by electron microscopy and RNA content. The other, the C fraction (C-antigen), contained low infectivity with little RNA and was similar to the structure of the heat-treated virus. As induction of neutralizing antibodies is associated with the immunization of intact virus structures (D-antigen) but not with the immunization of C-antigen viral preparations, the potency of IPV has been based on the D-antigen content. Thus, efficient in vitro methods for the D-antigen measurement are needed for screening stable vaccine formulations.

In this study, various surfactant-based formulations were screened for sIPV lyophilization, and size-exclusion high-performance liquid chromatography (SE-HPLC) [23] was implemented as a novel high-throughput formulation assay for D-antigen quantitation of sIPV. Finally, a room-temperature stable sIPV prepared by leading formulation induced strong neutralizing antibodies and full protection against wild-type poliovirus challenge in vivo. This sIPV formula will not only facilitate the vaccine distribution without the need of refrigeration but also contribute to the poliovirus endgame introduced by the Global Polio Eradication Initiative.

### Materials and Methods

### Cells and Viruses

Vero cells and HeLa cells were purchased from ATCC and maintained in Dulbecco's modified Eagle's medium (Thermo Fisher Scientific, #11965118) containing 10% Fetal Bovine Serum (VWR Life Science Seradigm, #1500-500) and 1% penicillin-streptomycin (Thermo Fisher Scientific, #15140163). Transient transfections for virus rescue were performed with polyethylenimine (PEI) transfection reagent (Polysciences, #23966) according to the manufacturer's instructions. Scale-up culturing of Vero cells were done in a 3 L spinner flask (Corning Life Sciences, #4502) after attaching Vero cells to micro carrier beads (GE Healthcare Life Sciences, #17044801). Media was changed every two days by removing half of the media and reintroducing fresh media.

Hela cells were co-transfected with pREV plasmid encoding T7 RNA polymerase and Sabin strain PV molecular clone (provided by Dr. Julie Pfeiffer from University of Texas Southwestern Medical Center) for 96 hrs with PEI at 3:1 ratio. When 90-95% CPE was confirmed after 96 hrs, cells were scraped off and supernatant was collected. The supernatant/cell mixtures were freeze-thawed three times to release the virion from the infected cells and passed through a 0.2 μM polyethersulfone (PES) filter (Nalgene, #566-0020). The clarified supernatants were loaded at a multiplicity of infection (MOI) of 10 into a dish of Vero cells and the cells and supernatants were collected after overnight incubation in 32° C. After the supernatants were clarified, the virus suspension was loaded at a MOI of 30 into a suspension of Vero cells in a spinner flask. After overnight incubation in 32° C., the supernatants were collected and freeze-thawed three times.

### Virus Purification and Inactivation (Procedure Summarized in FIG. 6).

Viral suspension was first clarified by centrifugation and passed through a 0.2 μM PES filter. The clarified suspension was concentrated in a labscale tangential flow filtration system (EMD Millipore, #XX42LSS11) using a biomax 100 kDa cartridge (EMD Millipore, #PXB100C50) at 20 psi input and 10 psi backpressure. Viral concentrate was loaded into a HiPrep Sephacryl S-200 HR column (GE Healthcare Life Sciences, #17116601) and run through a 20 mM sodium phosphate buffer pH 7.0 at 0.5 mL/min on a Duoflow Chromatography system (Bio-Rad, #7600037). The first 280 nm UV absorption peak was collected and loaded into a 5 mL HiTrap DEAE Fast Flow Column (GE Healthcare Life Sciences, #17-5154-01) using a 20 mM sodium phosphate buffer at pH 7.0, and the flow through show UV 280 nm absorption peak was collected (FIG. 1).

Methanol-free formaldehyde (Thermo Fisher Scientific, #28908) at a final concentration of 0.025% and M199 media (Sigma-Aldrich, M0650) was added into the purified PV suspension. The suspension was then incubated at 37° C. for 14 days. The suspension was passed through a 0.2 μM PES filter after one week to remove any aggregates. After 14 days, sodium bisulfite was added to neutralize the formaldehyde. The suspension was then dialyzed using a 10 kDa slide-a-lyzer cassette (Thermo Fisher Scientific, #66453) back into 20 mM sodium phosphate buffer and 25 mM NaCl using the manufacturer's instructions.

### Virus Titration and Enzyme Linked Immunosorbent Assay (ELISA) D-Antigen Unit Measurement

Ten-fold serial dilutions of virus inoculum was absorbed into the well of confluent Vero cells for 90 min in 37° C. After the removal of virus solution, cells were overlaid with 0.75% Avicel/DMEM and incubated in a humidified incubator at 37° C. and 5% CO₂ for 6 days. To visualize the plaques, the cells were fixed by 4% formaldehyde and stained by 0.2% crystal violet solution.

ELISA plates were coated with bovine serum anti-poliovirus (National Institute for Biological Standards and Control, #234) antibody at a concentration of 1:100. The sIPV was loaded at two-fold dilutions and a WHO standard IPV (National Institute for Biological Standards and Control, #12/104) was used to produce a standard curve. Mouse monoclonal antibody against type 1 PV (Abcam, #ab47802) diluted at 1:1000 was added for detection, and secondary anti-mouse HRP antibody (Cell Signaling Technology, #7076) diluted at 1:1000 was loaded. TMB solution (BD Biosciences, #555214) was used for quantification on a FilterMax F5 microplate reader (Molecular Devices, F5) after the reaction was stopped using hydrosulfuric acid.

### Size Exclusion—High Performance Liquid Chromatography (SE-HPLC)

SE-HPLC was performed with an Agilent 1100 series instrument (Agilent Technologies, #G1380-90000) equipped with quaternary pump, degasser, temperature controlled autosampler, a UV/Vis diode array detector (DAD) and an Agilent 1200 series fluorescence detector (FLD). TSKgel G6000PW_{XL} (7.8 mm×30 cm) or TSKgel G3000SW_{XL} (7.8 mm×30 cm) column purchased from Tosoh Bioscience (King of Prussia, Pa.) was used. At a flow rate of 0.8 mL/min, 100 μL of sample was injected per analysis. The mobile phase contained 50 mM Sodium Phosphate, 140 mM NaCl at pH 6.7. The FLD was used as the primary detector and set to acquire data at an excitation of 280 nm and an emission of 336 nm. For polio vaccine peak identification, the elution volume and the corresponding retention time of the FLD signal was calculated based on a standard calibration curve. To calculate polio vaccine recovery, the area under the curve of formulations before lyophilization was compared to the area under the curve of formulations after lyophilization.

### Dynamic Light Scattering (DLS) Analysis

DLS measurements to obtain the mean radius of IPV were performed with DynaPro Plate Reader (Wyatt Technology). IPV samples were prepared by diluting the vaccine 1:1 with phosphate buffer and filtered through 0.2 μm PES for DLS analysis. The samples were analyzed at 25 μL volume in triplicate and the mean particle radius for the vaccine was calculated via the Dynamics software (version 7.1.7).

### Lyophilized Cake Moisture Determination

The water (or moisture) content in solid lyophilized formulation was determined by coulometric titration using a Mettler C20 Coulometric Titrator (Mettler Toledo, #51105510) and an oven. Briefly, each lyophilized sample was brought to room temperature before caps were removed for analysis and subsequently heated in an oven. The moisture released from heating was carried from the oven to the Karl Fischer (KF) titration cell, which contained KF reagent for the reaction. Moisture in the titration cell was continuously titrated until an endpoint was reached. Each sample was measured in duplicate. Once an analysis was complete, results were generated automatically.

### Formulation Matrix

All formulations were prepared at twice the concentration to allow for a final 1:1 dilution by volume with the IPV sample. Each stock formulation was prepared by dissolving the stabilizers and bulking agents into a 10 mM Histidine buffer containing either polysorbate 20 or poloxamer 188. The adjustment of pH was performed with HCl prior to sterile filtration using 0.2 μm PES membrane. The final IPV-formulation solutions were prepared by mixing equal volumes of IPV sample and stock formulation to generate the formulation matrix for testing. Each mixture was filled into sterilized 2 cc glass vials (West Pharmaceutical Services) in two (2) replicates and half-capped with sterilized Diakyo 13 mm serum Flurotec stoppers (West Pharmaceutical Services). All sample preparation and the filling process were performed under aseptic conditions in a Class II biological safety cabinet.

Lyophilization Process Design of sIPV

For this study, all formulations were lyophilized using a conservative cycle designed to generate elegant lyophilized cakes with acceptable moisture content without compromising the vaccine product quality and integrity. The half-capped vials were loaded into a VirTis Genesis 25EL pilot lyophilizer (SP Scientific, #100001991) at a shelf temperature of 5° C. Following loading, the vials were slowly frozen to −50° C., held at the same temperature for 2 hrs and subsequently warmed to −15° C. at 0.5° C./min ramp rate. The vials were held at −15° C. for 2 hrs prior to initiating the primary drying step. The primary drying was performed at −15° C. shelf temperature with 100 mTorr chamber pressure for 10 hrs. The secondary drying step was designed to remove residual water that did not sublimate during the primary drying step; thus, the shelf temperature was increased to 25° C. and held for 3 hrs. After the completion of the lyophilization cycle, the vials were stoppered in a partial vacuum, labeled and stored at 2-8° C. before analysis.

### Animal Care

Transgenic Polio Virus Receptor (cPVR) Mice were a gift from Dr. Raul Andino (University of California, San Francisco) and were maintained in a USC mice facility according to the university's regulation for animal care and handling (IACUC).

### Poliovirus Challenge

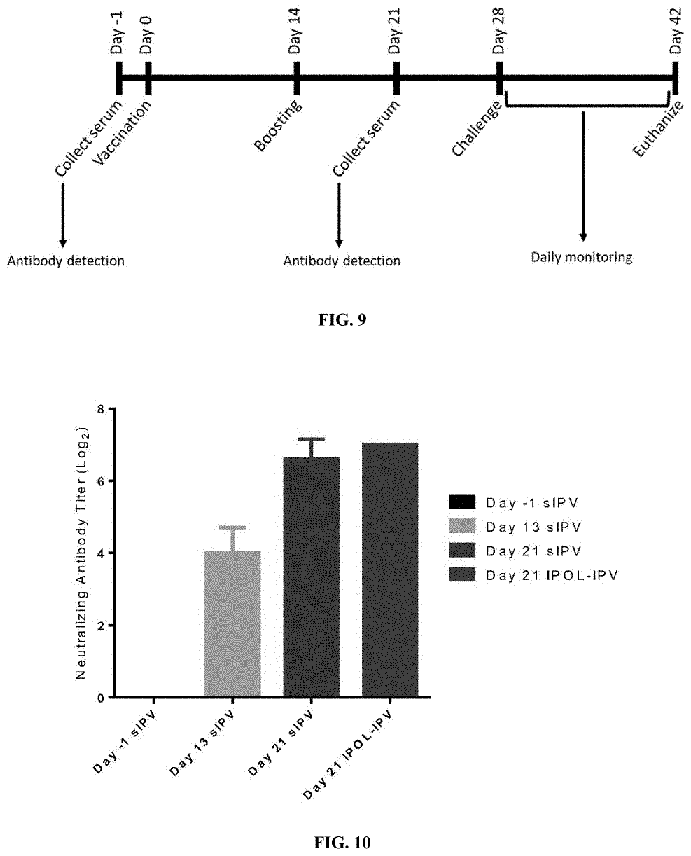

6-week-old cPVR mice (n=8), which confirmed the expression of PVR (data not shown), were vaccinated with half of a human dose of IPV (20 DU) via intraperitoneal (IP) route, and they were boosted with the same dose after 2 weeks. 14 days following the booster, the mice were injected with WT 1 Mahoney PV at a dose of 50 PD₅₀ (50% paralytic dose). Mice were monitored for two weeks using a blinded paralysis scoring system outlined by the WHO for mouse neurovirulence test for OPV (Vaccination and challenge procedure summarized in FIG. 9). If both legs dragged during ambulatory motions, legs hung when climbing across a rail, and they were unable to grip the rail, they were scored as paralyzed. If the mice maintained partial ability to move limbs forward, legs hung when climbing across a rail but recovered, but still unable to grip the rail, the mice were scored as paresis. Paresis mice received a paralyzed score if they showed signs of paresis for two consecutive days. Mice were euthanized after showing a score as paralyzed for a humane end point.

### Microneutralization Assay

Neutralization assay followed WHO standardized protocol for the assay [35, 51] with a little modification. Briefly, mice serum was collected by retro-orbital breeding on day −1 and day 21. The blood was allowed to clot at 4° C. for two days, centrifuged at 1,000×g for 30 min at 4° C. and the supernatant was collected. After heat-inactivation, sera were diluted in DMEM media in a two-fold serial dilution and an equal volume of 100 TCID₅₀ of WT Mahoney PV was added and incubated for 3 hrs in 37° C. These mixtures were then infected in about 80-90% confluent Vero cells plated in a 96-well plate in 4° C. for 18 hrs, washed with PBS and further incubated for 5 days in DMEM media. Neutralization antibody titers were calculated from dilutions that correspond to 50% reduction of virus infection compared to control.

### Results

sIPV Preparation

In order to prepare highly purified sIPV, Applicant followed the IPV production process previously published with modification (FIG. 6) [24]. Stock sIPV was generated from Hela cells by co-transfecting a cDNA plasmid encoding of Sabin poliovirus viral RNA and pREV encoding T7 RNA polymerase. Cultures were monitored until 90-95% CPE was observed [25, 26]. Viruses were then harvested by freezing and thawing the supernatants and cell mixtures, followed by filtration. This P0 virus stock was used as a working stock to scale up virus production in Vero cells using 3 L spinner flasks. 18 hours after infecting Vero cells with sIPV P0 stock at a MOI of 30, the supernatants were collected and freeze-thawed three times. To establish a vaccine production platform mimicking clinical use, Applicant used a multistep purification process including ultrafiltration, gel filtration and ion-exchange chromatography. Virus titers were checked at each step to ensure the optimal virus purification and to gain maximal virus recovery (FIG. 6). The purity of virus was confirmed by loading the SDS-treated virion on PAGE-gel, followed by silver staining (FIG. 1). Virus inactivation was carried out by using methanol-free formaldehyde at a final concentration of 0.025% to minimize the capsid protein alteration and then incubated at 37° C. After 14 days, sodium bisulfite was added to neutralize the formaldehyde. The suspension was then dialyzed using a 10 kDa slide-a-lyzer cassette against 20 mM sodium phosphate buffer and 25 mM NaCl according to the manufacturer's instructions.

Development of High-Throughput SE-HPLC Analysis of sIPV

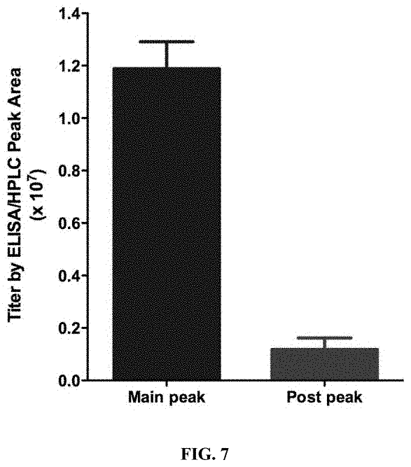

There are two distinct antigenic forms of PV: infectious virion particles are referred to as D-antigen (D-Ag) and non-infectious empty virion particles are referred to as C-antigen (C-Ag) [22]. Due to difference of the antigenic forms of D-Ag and C-Ag, only the D-Ag form of virion particles shows immunogenic response to viral infection. Moreover, D-Ag can be converted into C-Ag by heating at 56° C., thus C-Ag is also called H-antigen (H-Ag) [27]. The potency of IPVs has been determined by the amount of D-antigens present in the vaccine, typically by enzyme-linked immunosorbent assay (ELISA) [28]. Here, size-exclusion high-performance liquid chromatography (SE-HPLC) was investigated as a novel method for determining the antigenicity of sIPV through separation of intact viral particles from disintegrated capsid proteins based on hydrodynamic radius. SE-HPLC analysis of sIPV showed one main peak and one post peak (FIG. 2A), detecting the intrinsic fluorescence of tryptophan residues at λₑₓ/λₑₘ of 280/336 nm. A preliminary stability test was performed by storing the main peak eluate of sIPV at 4° C. for one week that was subsequently analyzed by SE-HPLC. These results revealed that the main peak degraded into the post peak species, and that all degradant species were detected in this method without loss in total area (FIG. 2B and Table 1). Dynamic light scattering (DLS) analysis revealed that the main peak observed by SE-HPLC contained a mono-dispersed particle of 14.6 nm in radius (Table 2) which matched closely with the PV radius of ˜15 nm [1]. Eluates of the main and post peaks from SE-HPLC (FIG. 2A) were both collected and measured for the D-AgU by ELISA [29]. This also confirmed that only the main peak showed reactivity with the type 1 PV antibody (FIG. 7). These results strongly demonstrated the utility of SE-HPLC as a reliable and efficient method for measuring stability of sIPV in various lyophilized formulations.

### Surfactant-Based Formulation Buffer for Lyophilization

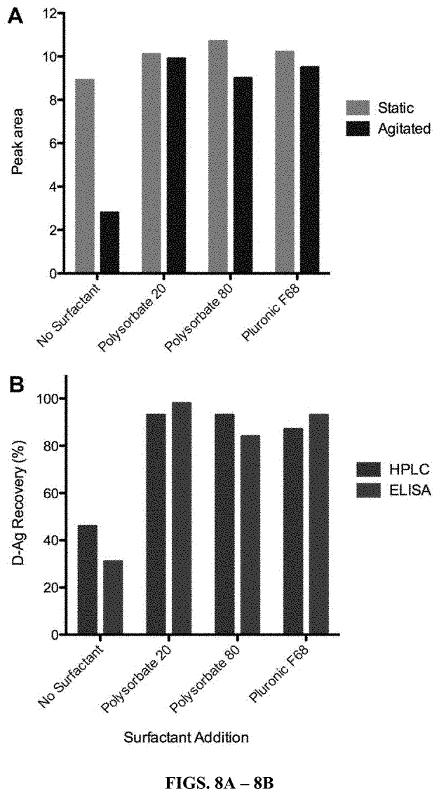

A desirable lyophilized formulation focused on the following attributes: minimal loss of D-Ag, formation of an elegant cake structure correlating with good product integrity [30], and stability following storage at ambient temperature. A number of traditional stabilizers and lyophilic excipients including glycine, mannitol, sorbitol, sucrose, and magnesium sulfate were evaluated, and formulations maintaining D-Ag recovery rates greater than 80% upon lyophilization were selected for further optimization (Table 3). Following the primary round of screening, the stabilizing effect of surfactants [21] was assessed by agitating the samples for 4 hrs with and without the addition of 0.01% polysorbate 20, 0.01% polysorbate 80, or 0.1% pluronic. While agitation significantly reduced D-antigen levels, the addition of polysorbate 20 or pluronic F68 effectively mitigated D-Ag loss (FIG. 8A, FIG. 8B). Magnesium ion at 1 mM concentration was also considered as a stabilizer based on its stabilizing effect on other vaccines including OPV and IPV [20]. Formulation pHs ranging from 6 to 8 were tested, but optimal D-Ag recovery was observed at a neutral pH. A histidine buffer used to maintain the neutral pH was combined with mannitol as a bulking agent and sucrose or sorbitol as a stabilizing sugar (lyophilization condition summarized in Table 5) [21]. This formulation also showed low values of moisture content by Karl Fischer analysis of 0.77 to 1.30% (Table 4) [17].

Thermostability of Lyophilized sIPV and Vaccine Efficacy In Vivo

To test the thermostability of the leading lyophilized formulation candidates (Formulation code of F4, F8 and F9 as summarized in Table 3), sIPVs in the formulation were incubated after lyophilization at 4° C. (FIG. 3A), 25° C. (FIG. 3B), or 40° C. (FIG. 3C) for up to 4 weeks. Overall, the leading formulation was 10 mM histidine, 5% mannitol, 1 mM MgSO4, 1% sorbitol, and 0.5% pluronic F68 at pH 7 (Formulation code, F4), with the most efficient in D-Ag recovery of 96%, 90% and 83% at 4° C., 25° C., and 40° C., respectively. This F4 formulation was selected for in vivo protective efficacy testing against wild-type (WT) PV infection [31].

A total of six groups of poliovirus receptor transgenic (cPVR) mice (n=8) expressing human CD155 for viral entry [32, 33] were vaccinated with 20 D-AgU of sIPV, lyophilized (lyo) sIPV, commercial IPOL-IPV or PBS incubated for 4 weeks in indicated temperatures. cPVR mice were vaccinated, boosted, challenged and observed for two weeks for signs of paralysis (FIG. 9), using a blinded scoring method outlined in the WHO standard operating procedure for OPV neurovirulence testing [34]. For the serum neutralizing titers, Applicant first checked day −1 serum and confirmed that no mice were seroconverted as neutralization titer was below the detection limit (FIG. 10). Then, Applicant checked the neutralization titers on days 13 and 21 and found that the neutralization titers on day 13 were approximately two logs lower than those on day 21 (FIG. 10). Based on the WHO standardized in vivo potency testing of IPV in rodents along with the results of FIG. 10, day 21 after vaccination was determined as a time-point for the neutralization assay. These assays showed that lyophilized sIPV incubated at either 4° C. or 37° C. for 4 weeks developed similar neutralizing antibody titers to sIPV incubated at 4° C. (FIG. 4A). By striking contrast, sIPV incubated at 37° C. for 4 weeks showed considerable instability: it developed approximately a 7-fold reduced neutralizing antibody titer compared to sIPV incubated at 4° C. or lyophilized sIPV at either 4° C. or 37° C. for 4 weeks (FIG. 4A). Following the boost, cPVR mice were challenged with a 50% paralytic dose (50% PD₅₀) of WT Mahoney strain PV. This showed that lyophilized sIPV incubated at 4° C. or 37° C. for 4 weeks were able to protect mice from paralysis as strongly as commercial IPOL-IPV (FIG. 4B). This unambiguously demonstrates that the lyophilized sIPV remains stable after four weeks of incubation at 37° C. and induces strong neutralizing antibodies and full protection of poliovirus receptor transgenic mice against the in vivo challenge of wild-type poliovirus.

### Discussion

A majority of human vaccines are temperature-sensitive. The cold-chain dependence of current vaccines, which prevents exposure to ambient temperature and also to freezing [12], presents many obstacles that can lead to failure of vaccination campaigns. As previously reported [36], nearly 1.5 million children lose their lives due to vaccine-preventable diseases. Pharmaceutical Commerce reported that $12.5 billion was spent on cold-chain logistics, of which $9.1 billion was for cold-chain transportation and $3.4 billion was for specialized packaging and instrumentation. Thus, improving methods to generate thermostabilized vaccines can reduce the number of deaths caused by vaccine preventable diseases, and cut down on the expenditure used for cold-chain transport.

This study shows the establishment and optimization of lyophilization conditions to increase the in vitro and in vivo thermostability and vaccine capacity of sIPV at temperatures up to 40° C. for at least one month. The use of SE-HPLC enabled the analysis of various formulations as Applicant was able to distinguish between D-Ag and C-Ag by SE-HPLC, which was later confirmed by ELISA (FIG. 7) and DLS analysis (Table 2) [37]. Recent studies have shown the use of SE-HPLC for stability and potency testing assays for human papillomavirus vaccine [38], characterization of influenza vaccine constituents [39], and quality control of vaccines by characterizing the assembly of antigens [40]. In agreement with earlier studies using SE-HPLC, this method provides an effective means to screen the vaccine stability and antigen recovery after lyophilization in a high-throughput manner [23] compared to conventional ELISA [29].

In virus purification step, Applicant used tangential flow filtration (TFF), size-exclusion chromatography (SEC), and ion-exchange chromatography (IEC), followed by protein silver staining to ensure the high quality of poliovirus purification. The icosahedral poliovirus nucleocapsid is composed of 60 copies each of four coat proteins, VP1, VP2, VP3 and VP4 (1, 41). As VP4 protein is very small (˜7 k) and myristoylated, it migrated as broad bands in SDS gel and did not be clearly visualized (FIG. 1). However, the calculation of the band density and molecular weight of each VP protein showed that the level of VP4 protein in purified virions was similar to or slightly lower than those of the rest of VP1-3 proteins. It should be noted that while the D-antigen of poliovirus vaccine carries all four VP1-4 capsid proteins, only the VP1 capsid protein is responsible for the generation of protective immune response against wild-type virus infection [43].

Applicant screened the optimal lyophilization formulation for minimal D-Ag loss, elegant cake structure reflecting good product integrity, and stability upon storage at ambient temperature. In order to achieve a successful lyophilized poliovirus vaccine for this study, various excipients including glycine, mannitol, sorbitol, sucrose, and magnesium sulphate at varying concentrations and combinations were screened to determine their effectiveness as a lyoprotectant during lyophilization and titer recoveries were calculated as normalized results from pre-lyophilized liquid formulations. Our leading formulation, containing 10 mM histidine, 5% mannitol, 1 mM MgSO₄, 1% sorbitol, and 0.5% pluronic F68 at pH 7, resulted in higher D-Ag recovery following lyophilization and subsequent ambient temperature storage than previous lyophilization of polio vaccine [17]. Agitation studies also showed a clear benefit of surfactants such as pluronic F68 or polysorbate 20 for the stability from physical stress (FIG. 8A, FIG. 8B), which is an important factor to be considered during vaccine manufacture [44, 45]. Finally, the moisture content of our leading formulation was only 0.77%, which is considerably lower than previous polio vaccine lyophilization attempts [16]. Thus, Applicant's formulation provides an optimal condition for the stability of sIPV during lyophilization and ambient temperature storage.

Adopting the vaccination/boosting regimen of previously reported [46-48], Applicant showed the thermostable lyophilized sIPV incubated at 37° C. for 4 weeks induced a potent anti-poliovirus immune response in cPVR mice and effectively protected these mice from challenge with WT PV Mahoney strain. Moreover, the level of type 1 PV neutralizing antibodies of mice vaccinated with the F4 formulated sIPV were similar to levels of commercial IPOL vaccine. A recent study by Tzeng and colleagues has shown injectable microparticle system that releases multiple pulses of antigen over time. Their lead formulation also releases two pulses of antigen one month apart, mimicking vaccination/boosting regimen that is being used in the developing world [49]. Although the lyophilization formulation removes any transportation complications due to its long-term stability, Applicant observed that the D-AgU slightly decreased after 4 weeks of incubation at 25° C. Maintaining a consistent D-AgU for the duration of the month will be paramount to vaccine stockpiles.

The typical maximum amount of time that the vaccine vial is stored at health posts is 3 months. Karp and colleagues [50] have suggested that if the vaccine is stable for more than 2 months, it is possible to remove cold-chain equipment at health posts and stockpile the vaccines. If the vaccine acquires more than 12 months thermostability, it enables the removal of cold-chain equipment at every check points and redesign the supply chain structure. To address this issue, further optimization experiments are in progress to monitor the vaccine thermostability up to 3-month and 12-month time points. Therefore, further optimization will hopefully achieve a “truly” temperature stable polio vaccine. With the endgame of polio eradication in sight, the shift from OPV to IPV will become a necessity to avoid manufacture-borne polio infections. Our study demonstrates that the thermostable lyophilized sIPV induces potent anti-poliovirus antibodies in cPVR mice and effectively protects mice from WT PV infection. Overall, this novel approach for high-throughput evaluation of antigenicity provides a means to accelerate the process of thermostable vaccine development and facilitate the availability and efficacy of vaccinations around the world.

TABLE 1
Integration results of chromatograms shown in FIG. 2.

| | SEC-HPLC Peak Areas | | |
|---|---|---|---|
| | (Fluorescence Intensity) | | Total |
| Sample | Main Peak | Post Peak | Area |
| Sabin inactivated poliovirus | 11.8 | 3.7 | 15.5 |
| control | | | |
| After 1 week at 4° C. | 1.8 | 14.1 | 15.9 |

TABLE 2
Dynamic light scattering analysis of main peak.

| Peak | Parameters | Analysis |
|---|---|---|
| Main peak | Mean radius (nm) | 14.6 |
| | % Pd | 8.1 |
| | % Intensity | 100.0 |
| | % Mass | 100.0 |

TABLE 3
List of candidate formulations for sIPV lyophilization and
percent recovery of D-antigen following lyophilization.

| | | | | | | | | ELISA |
|---|---|---|---|---|---|---|---|---|
| Formulation | Bulking | | | | | | [symbol] SE-HPLC | Recovery |
| Code | Agent/stabilizer | Buffer | Sugar | pH | Surfactant | Stabilizer | Re [symbol] | (%) |
| F1 | 2.5% | 10 mM | 1% | 8 | 0.1% Pluronic F68 | 1 mM MgSO4 | [symbol] 8 | 85 |
| F2 | 5% | 10 mM | 1% | 7 | None | 1 mM MgSO4 | [symbol] 1 | 19 |
| F3 | 5% | 10 mM | 1% | 7 | 0.1% Pluronic F68 | 1 mM MgSO4 | [symbol] 7 | 78 |
| F4 | 5% | 10 mM | 1% | 7 | 0.5% Pluronic F68 | 1 mM MgSO4 | [symbol] 9 | 95 |
| F5 | 5% | 10 mM | 1% | 7 | 1.0% Pluronic F68 | 1 mM MgSO4 | [symbol] 9 | 91 |
| F6 | 5% | 10 mM | 1% | 7 | 0.1% Pluronic F68 | 1 mM MgSO4 | [symbol] 6 | 88 |
| F7 | 5% | 10 mM | 1% | 7 | 0.5% Pluronic F68 | 1 mM MgSO4 | [symbol] 7 | 73 |
| F8 | 5% | 10 mM | 1% | 7 | 0.01% Polysorbate | 1 mM MgSO4 | [symbol] 9 | 90 |
| F9 | 5% | 10 mM | 1% | 7 | 0.01% Polysorbate | 1 mM MgSO4 | [symbol] 9 | 91 |
| F10 | 5% | 10 mM | 1% | 7 | 0.1% Polysorbate 20 | 1 mM MgSO4 | [symbol] 7 | 81 |
| F11 | 5% | 10 mM | 1% | 7 | 0.5% Polysorbate 20 | 1 mM MgSO4 | [symbol] 7 | 78 |
| F12 | 5% | 10 mM | 1% | 7 | 0.05% Polysorbate | 1 mM MgSO4 | [symbol] 7 | 82 |
| F13 | 5% | 10 mM | 1% | 7 | 0.1% Polysorbate 20 | 1 mM MgSO4 | [symbol] 8 | 74 |
| F14 | 2.5% | 10 mM | 1% | 7 | 0.1% Pluronic F68 | 1 mM MgSO4 | [symbol] 4 | 76 |
| F15 | 2.5% | 10 mM | 1% | 6 | 0.1% Pluronic F68 | 1 mM MgSO4 | [symbol] 5 | N.T.* |
| F16 | 2.5% | 10 mM | 1% | 6 | 0.1% Pluronic F68 | 1 mM MgSO4 | [symbol] 5 | N.T. |
| F17 | 5% | 10 mM | 1% | 6 | 0.1% Pluronic F68 | 1 mM MgSO4 | [symbol] 6 | N.T. |
| F18 | 2.5% | 10 mM | 1% | 6 | 0.1% Pluronic F68 | 1 mM MgSO4 | [symbol] 6 | N.T. |

*Not tested [symbol]
indicates data missing or illegible when filed

TABLE 4
Moisture content measurements of leading candidates.

| | % Moisture content |
|---|---|
| Formulation code | T = 0 |
| 1% water | 0.97 |
| F4 | 0.77 |
| F8 | 0.97 |
| F9 | 1.3 |

TABLE 5
Parameters for lyophilization cycle.

| | | | | Chamber |
|---|---|---|---|---|
| | | Time | Ramp Rate | Pressure |
| Step | Temperature | (min) | (° C./min) | (mT) |
| Loading | 5° C. | N/A | N/A* | N/A |
| Freezing | 5° C. to −50° C. | 110 min | 0.5° C./min | N/A |
| | −50° C. | 120 min | N/A | N/A |
| | −50° C. to −15° C. | 70 min | 0.5° C./min | N/A |
| | −15° C. | 120 min | N/A | N/A |
| Primary Drying | −15° C. | 700 min | N/A | 100 mT |
| Secondary | −30° C. to 25° C. | 80 min | 0.5° C./min | 100 mT |
| Drying | 25° C. | 120 min | N/A | 100 mT |

*Not applicable

### Equivalents

Additional aspects of this disclosure are provided in the appendix which is incorporated by reference herein.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

Additional information regarding the claimed embodiments are provided in the Appendices as attached hereto.

### EXPERIMENT NO. 1 REFERENCES

1. Bakker W A, Thomassen Y E, van't Oever A G, Westdijk J, van Oijen M G, et al. Inactivated polio vaccine development for technology transfer using attenuated Sabin poliovirus strains to shift from Salk-IPV to Sabin-IPV. Vaccine. 2011 Sep. 22; 29(41):7188-96. PubMed PMID: 21651934.
2. Kumru O S, Joshi S B, Smith D E, Middaugh C R, Prusik T, et al. Vaccine instability in the cold chain: mechanisms, analysis and formulation strategies. Biologicals. 2014 September; 42(5):237-59. PubMed PMID: 24996452.
3. Kraan H, van Herpen P, Kersten G, Amorij J P. Development of thermostable lyophilized inactivated polio vaccine. Pharm Res. 2014 October; 31(10):2618-29. PubMed PMID: 24760448; PubMed Central PMCID: PMC4197379.
4. Global Polio Eradication Initiative. This Week. http://polioeradication.org/polio-today/polio-now/this-week/
5. World Health Organization (2016). Preparing for the withdrawal of all oral polio vaccines. WHO
6. World Health Organization. Polio vaccines: WHO position paper, March 2016-recommendations. Vaccine. 2017 Mar. 1; 35(9):1197-1199. PubMed PMID: 27894720.
7. Wood D J, Sutter R W, Dowdle W R. Stopping poliovirus vaccination after eradication: issues and challenges. Bull World Health Organ. 2000; 78(3):347-57. PubMed PMID: 10812731; PubMed Central PMCID: PMC2560716.
8. Fox H, Knowlson S, Minor P D, Macadam A J. Genetically Thermo-Stabilised, Immunogenic Poliovirus Empty Capsids; a Strategy for Non-replicating Vaccines. PLoS Pathog. 2017 January; 13(1):e1006117. PubMed PMID: 28103317; PubMed Central PMCID: PMC5245777.
9. Chan H T, Xiao Y, Weldon W C, Oberste S M, Chumakov K, et al. Cold chain and virus-free chloroplast-made booster vaccine to confer immunity against different poliovirus serotypes. Plant Biotechnol J. 2016 November; 14(11):2190-2200. PubMed PMID: 27155248; NIHMSID: NIHMS785245; PubMed Central PMCID: PMC5056803.
10. Tzeng S Y, Guarecuco R, McHugh K J, Rose S, Rosenberg E M, et al. Thermostabilization of inactivated polio vaccine in PLGA-based microspheres for pulsatile release. J Control Release. 2016 Jul. 10; 233:101-13. PubMed PMID: 27178811; PubMed Central PMCID: PMC4925381.
11. Hansen L J J, Daoussi R, Vervaet C, Remon J P, De Beer T R M. Freeze-drying of live virus vaccines: A review. Vaccine. 2015 Oct. 13; 33(42):5507-5519. PubMed PMID: 26364685.
12. Galazka A, Milstein J, Zaffra M. Thermostability of vaccines. World Health Organization
13. Wilton T, Dunn G, Eastwood D, Minor P D, Martin J. Effect of formaldehyde inactivation on poliovirus. J Virol. 2014 October; 88(20):11955-64. PubMed PMID: 25100844; PubMed Central PMCID: PMC4178759.
14. Thomassen Y E, van't Oever A G, Vinke M, Spiekstra A, Wijffels R H, et al. Scale-down of the inactivated polio vaccine production process. Biotechnol Bioeng. 2013 May; 110(5):1354-65. PubMed PMID: 23192424.
15. Thomassen Y E, van't Oever A G, van Oijen M G, Wijffels R H, van der Pol L A, et al. Next generation inactivated polio vaccine manufacturing to support post polio-eradication biosafety goals. PLoS One. 2013; 8(12):e83374. PubMed PMID: 24349497; PubMed Central PMCID: PMC3861478.
16. Hogle J M, Chow M, Filman D J. Three-dimensional structure of poliovirus at 29 A resolution. Science. 1985 Sep. 27; 229(4720):1358-65. PubMed PMID: 2994218.
17. Shimizu H. Development and introduction of inactivated poliovirus vaccines derived from Sabin strains in Japan. Vaccine. 2016 Apr. 7; 34(16):1975-85. PubMed PMID: 25448090.
18. Crotty S, Hix L, Sigal L J, Andino R. Poliovirus pathogenesis in a new poliovirus receptor transgenic mouse model: age-dependent paralysis and a mucosal route of infection. J Gen Virol. 2002 July; 83(Pt 7):1707-20. PubMed PMID: 12075090.
19. Taffs R E, Chernokhvostova Y V, Dragunsky E M, Nomura T, Hioki K, et al. Inactivated poliovirus vaccine protects transgenic poliovirus receptor mice against type 3 poliovirus challenge. J Infect Dis. 1997 February; 175(2):441-4. PubMed PMID: 9203668.
20. Dragunsky E M, Ivanov A P, Abe S, Potapova S G, Enterline J C, et al. Further development of a new transgenic mouse test for the evaluation of the immunogenicity and protective properties of inactivated poliovirus vaccine. J Infect Dis. 2006 Sep. 15; 194(6):804-7. PubMed PMID: 16941347.
21. Martin J, Crossland G, Wood D J, Minor P D. Characterization of formaldehyde-inactivated poliovirus preparations made from live-attenuated strains. J Gen Virol. 2003 July; 84(Pt 7):1781-8. PubMed PMID: 12810872.
22. WHO (2012) Standard Operating Procedure Neurovirulence test of Types 1, 2 or 3 Live Attenuated Poliomyelitis Vaccines (Oral) in transgenic mice susceptible to poliovirus. WHO.
23. Wood D J, Heath A B. Collaborative study for the establishment of a rat bioassay for inactivated poliovaccine. Pharmeuropa special issue BIO 2000-1:25-49.
24. Longo, P. A., Kavran, J. M., Kim, M.-S., & Leahy, D. J. (2013). Transient Mammalian Cell Transfection with Polyethylenimine (PEI). Methods in Enzymology, 529, 227-240. http://doi.org/10.1016/B978-0-12-418687-3.00018-5.
25. Sawyer L A, McInnis J, Albrecht P. Quantitation of D antigen content in inactivated poliovirus vaccine derived from wild-type or sabin strains. Biologicals. 1993 June; 21(2):169-77. PubMed PMID: 8297601.
26. Longo, P. A., Kavran, J. M., Kim, M.-S., & Leahy, D. J. (2013). Transient Mammalian Cell Transfection with Polyethylenimine (PEI). Methods in Enzymology, 529, 227-240. http://doi.org/10.1016/B978-0-12-418687-3.00018-5.

### EXPERIMENT NO. 2 REFERENCES

1. Hogle J M, Chow M, Filman D J. 1985. Three-dimensional structure of poliovirus at 2.9 A resolution. Science 229:1358-1365.
2. Kitamura N, Semler B L, Rothberg P G, Larsen G R, Adler C J, Dorner A J, Emini E A, Hanecak R, Lee J J, van der Werf S, Anderson C W, Wimmer E. 1981. Primary structure, gene organization and polypeptide expression of poliovirus RNA. Nature 291:547-553.
3. Rappuoli R, Pizza M, Del Giudice G, De Gregorio E. 2014. Vaccines, new opportunities for a new society. Proc Natl Acad Sci USA 111:12288-12293.
4. Barquet N, Domingo P. 1997. Smallpox: the triumph over the most terrible of the ministers of death. Ann Intern Med 127:635-642.
5. Plotkin S. 2014. History of vaccination. Proc Natl Acad Sci USA 111:12283-12287.
6. Salk J E. 1955. Considerations in the preparation and use of poliomyelitis virus vaccine. J Am Med Assoc 158:1239-1248.
7. Krugman S, Warren J, Eiger M S, Berman P H, Michaels R M, Sabin A B. 1961. Immunization with live attenuated poliovirus vaccine. Am J Dis Child 101:23-29.
8. Ghendon Y, Robertson S E. 1994. Interrupting the transmission of wild polioviruses with vaccines: immunological considerations. Bull World Health Organ 72:973-983.
9. Platt L R, Estivariz C F, Sutter R W. 2014. Vaccine-associated paralytic poliomyelitis: a review of the epidemiology and estimation of the global burden. J Infect Dis 210 Suppl 1:S380-389.
10. Jenkins H E, Aylward R B, Gasasira A, Donnelly C A, Mwanza M, Corander J, Gamier S, Chauvin C, Abanida E, Pate M A, Adu F, Baba M, Grassly N C. 2010. Implications of a circulating vaccine-derived poliovirus in Nigeria. N Engl J Med 362:2360-2369.
11. Friedrich M J. 2016. Switch from trivalent to bivalent oral polio vaccine. JAMA 315:2513-2513.
12. Kartoglu U, Milstien J. 2014. Tools and approaches to ensure quality of vaccines throughout the cold chain. Expert Rev Vaccines 13:843-854.
13. Chen D, Kristensen D. 2009. Opportunities and challenges of developing thermostable vaccines. Expert Rev Vaccines 8:547-557.
14. Wang G, Wang H J, Zhou H, Nian Q G, Song Z, Deng Y Q, Wang X, Zhu S Y, Li X F, Qin C F, Tang R. 2015. Hydrated silica exterior produced by biomimetic silicification confers viral vaccine heat-resistance. ACS Nano 9:799-808.
15. Tzeng S Y, Guarecuco R, McHugh K J, Rose S, Rosenberg E M, Zeng Y, Langer R, Jaklenec A. 2016. Thermostabilization of inactivated polio vaccine in PLGA-based microspheres for pulsatile release. J Control Release 233:101-113.
16. Kraan H, Ploemen I, van de Wijdeven G, Que I, Lowik C, Kersten G, Amorij J P. 2015. Alternative delivery of a thermostable inactivated polio vaccine. Vaccine 33:2030-2037.
17. Kraan H, van Herpen P, Kersten G, Amorij J P. 2014. Development of thermostable lyophilized inactivated polio vaccine. Pharm Res 31:2618-2629.
18. Adams G. 2007. The principles of freeze-drying. Methods Mol Biol 368:15-38.
19. Fox H, Knowlson S, Minor P D, Macadam A J. 2017. Genetically Thermo-Stabilised, Immunogenic Poliovirus Empty Capsids; a Strategy for Non-replicating Vaccines. PLoS Pathog 13:e1006117.
20. World Health Organization. 2017. Polio vaccines: WHO position paper, March 2016-recommendations. Vaccine 35:1197-1199.
21. Adams G D J. 2003. Lyophilization of Vaccines, p 223-243. In Robinson A, Hudson M J, Cranage M P (ed), Vaccine Protocols doi:10.1385/1-59259-399-2:223. Humana Press, Totowa, N.J.
22. Mayer M M, Rapp H J, Roizman B, Klein S W, Cowan K M, Lukens D. 1957. The purification of poliomyelitis virus as studied by complement fixation. J Immunol 78:435-455.
23. Schrag D, Corbier M, Raimondi S. 2014. Size exclusion-high-performance liquid chromatography (SEC-HPLC). Methods Mol Biol 1131:507-512.
24. Thomassen Y E, van't Oever A G, Vinke M, Spiekstra A, Wijffels R H, van der Pol L A, Bakker W A. 2013. Scale-down of the inactivated polio vaccine production process. Biotechnol Bioeng 110:1354-1365.
25. Bouchard M J, Lam D H, Racaniello V R. 1995. Determinants of attenuation and temperature sensitivity in the type 1 poliovirus Sabin vaccine. J Virol 69:4972-4978.
26. Pfeiffer J K, Kirkegaard K. 2003. A single mutation in poliovirus RNA-dependent RNA polymerase confers resistance to mutagenic nucleotide analogs via increased fidelity. Proc Natl Acad Sci USA 100:7289-7294.
27. Hummeler K, Hamparian V V. 1958. Studies on the complement fixing antigens of poliomyelitis. I. Demonstration of type and group specific antigens in native and heated viral preparations. J Immunol 81:499-505.
28. Wilton T, Dunn G, Eastwood D, Minor P D, Martin J. 2014. Effect of formaldehyde inactivation on poliovirus. J Virol 88:11955-11964.
29. Lin A V. 2015. Indirect ELISA. Methods Mol Biol 1318:51-59.
30. Adams G D, Cook I, Ward K R. 2015. The principles of freeze-drying. Methods Mol Biol 1257:121-143.
31. Shimizu H. 2016. Development and introduction of inactivated poliovirus vaccines derived from Sabin strains in Japan. Vaccine 34:1975-1985.
32. Ren R B, Costantini F, Gorgacz E J, Lee J J, Racaniello V R. 1990. Transgenic mice expressing a human poliovirus receptor: a new model for poliomyelitis. Cell 63:353-362.
33. Crotty S, Hix L, Sigal L J, Andino R. 2002. Poliovirus pathogenesis in a new poliovirus receptor transgenic mouse model: age-dependent paralysis and a mucosal route of infection. J Gen Virol 83:1707-1720.
34. Dragunsky E, Nomura T, Karpinski K, Furesz J, Wood D J, Pervikov Y, Abe S, Kurata T, Vanloocke O, Karganova G, Taffs R, Heath A, Ivshina A, Levenbook I. 2003. Transgenic mice as an alternative to monkeys for neurovirulence testing of live oral poliovirus vaccine: validation by a WHO collaborative study. Bull World Health Organ 81:251-260.
35. Wood D J, Heath A B, Sawyer L A. 1995. A WHO Collaborative study on assays of the antigenic content of inactivated poliovirus vaccines. Biologicals 23:83-94.
36. Ashok A, Brison M, LeTallec Y. 2017. Improving cold chain systems: Challenges and solutions. Vaccine 35:2217-2223.
37. Al-Ghobashy M A, Mostafa M M, Abed H S, Fathalla F A, Salem M Y. 2017. Correlation between Dynamic Light Scattering and Size Exclusion High Performance Liquid Chromatography for Monitoring the Effect of pH on Stability of Biopharmaceuticals. J Chromatogr B Analyt Technol Biomed Life Sci 1060:1-9.
38. Mostafa M M, Al-Ghobashy M A, Fathalla F A, Salem M Y. 2016. Immunoaffinity extraction using conformation-dependent antibodies coupled to SE-HPLC for the development of stability and potency-indicating assay for quadrivalent human papillomavirus vaccine. J Chromatogr B Analyt Technol Biomed Life Sci 1032:211-217.
39. Garcia-Canas V, Lorbetskie B, Cyr T D, Hefford M A, Smith S, Girard M. 2010. Approach to the profiling and characterization of influenza vaccine constituents by the combined use of size-exclusion chromatography, gel electrophoresis and mass spectrometry. Biologicals 38:294-302.
40. Yang Y, Li H, Li Z, Zhang Y, Zhang S, Chen Y, Yu M, Ma G, Su Z. 2015. Size-exclusion HPLC provides a simple, rapid, and versatile alternative method for quality control of vaccines by characterizing the assembly of antigens. Vaccine 33:1143-1150.
41. Hogle J M, Chow M, Filman D J. 1987. The structure of poliovirus. Sci Am 256:42-49.
42. Wood D J, Heath A B, Kersten G F, Hazendonk T, Lantinga M, Beuvery E C. 1997. A new WHO International Reference Reagent for use in potency assays of inactivated poliomyelitis vaccine. Biologicals 25:59-64.
43. Chow M, Baltimore D. 1982. Isolated poliovirus capsid protein VP1 induces a neutralizing response in rats. Proc Natl Acad Sci USA 79:7518-7521.
44. Kumru O S, Joshi S B, Smith D E, Middaugh C R, Prusik T, Volkin D B. 2014. Vaccine instability in the cold chain: mechanisms, analysis and formulation strategies. Biologicals 42:237-259.
45. Burke C J, Hsu T A, Volkin D B. 1999. Formulation, stability, and delivery of live attenuated vaccines for human use. Crit Rev Ther Drug Carrier Syst 16:1-83.
46. Baca-Estrada M, Griffiths E. 2006. Regulation and standardization of IPV and IPV combination vaccines. Biologicals 34:159-161.
47. Taffs R E, Chernokhvostova Y V, Dragunsky E M, Nomura T, Hioki K, Beuvery E C, Fitzgerald E A, Levenbook I S, Asher D M. 1997. Inactivated poliovirus vaccine protects transgenic poliovirus receptor mice against type 3 poliovirus challenge. J Infect Dis 175:441-444.
48. Kouiavskaia D, Collett M S, Dragunsky E M, Sarafanov A, Chumakov K M. 2011. Immunogenicity of inactivated polio vaccine with concurrent antiviral V-073 administration in mice. Clin Vaccine Immunol 18:1387-1390.
49. Tzeng S Y, McHugh K J, Behrens A M, Rose S, Sugarman J L, Ferber S, Langer R, Jaklenec A. 2018. Stabilized single-injection inactivated polio vaccine elicits a strong neutralizing immune response. Proc Natl Acad Sci USA 115:E5269-E5278.
50. Karp C L, Lans D, Esparza J, Edson E B, Owen K E, Wilson C B, Heaton P M, Levine O S, Rao R. 2015. Evaluating the value proposition for improving vaccine thermostability to increase vaccine impact in low and middle-income countries. Vaccine 33:3471-3479.
51. Wood D J, Health, A. B. 2000. Collaborative study for the establishment of a rat bioassay for inactivated poliomyelitis vaccine. Pharmeuropa 1:25-49.

What is claimed is:

1. A vaccine formulation having a pH of from about 5.5 to about 8.5, comprising:
a. an effective amount of a virus or viral antigen;
b. from about 2% to about 10% of a bulking agent;
c. from about 5 mM to about 25 mM of a buffer;
d. from about 0.5% to about 1.5% of a sugar composition; and
e. from about 0.1% to about 10% or from about 0.5 mM to about 1.5 mM of a stabilizer.

2. The vaccine formulation of claim 1, further comprising from about 0.005% to about 1.5% of a surfactant solution.

3. The vaccine formulation of claim 1, further comprising from about 0.5 mM to about 1.5 mM of a metal ion.

4. The vaccine formulation of claim 1, wherein the virus or viral antigen comprises an effective amount of D-antigen units of formaldehyde-inactivated polio virus or viral antigen.

5. The vaccine formulation of claim 1, wherein the virus or viral antigen comprises an effective amount of D-antigen units of a heat-inactivated polio virus or viral antigen.

6. The vaccine formulation of claim 5, wherein the effective amount of the formaldehyde-inactivated polio virus is from about 0.3×10⁷ to about 10.0×10⁷ pfu/ml.

7. The vaccine formulation of claim 5, wherein the effective amount of the vaccine or antigen comprises from about 20 D-antigen Units/ml to about 100 D-antigen Units/ml.

8. The vaccine formulation of claim 5, wherein the effective amount of the virus or viral antigen comprises about 80 D-antigen Units/ml.

9. A vaccine formulation as set forth in Table 3.

10. The vaccine formulation of claim 9, wherein the effective amount of the vaccine or antigen comprises at least about 15% D-antigen recovery as measured using size-exclusion high-performance liquid chromatography (SE-HPLC) or an equivalent thereof.

11. The vaccine formulation of claim 9, wherein the effective amount of the vaccine or antigen comprises at least about 15% D-antigen recovery as measured using enzyme-linked immunosorbent assay (ELISA) or an equivalent thereof.

12. A lyophilized formulation of the vaccine formulation of claim 1.

13. The vaccine formulation of claim 12, wherein the lyophilized vaccine formulation has a moisture content of no more than about 2% water or an equivalent thereof.

14. A composition comprising an effective amount of the lyophilized formulation of claim 12 and a pharmaceutically acceptable carrier.

15. A kit comprising one or more of the vaccine formulation of claim 1, and instructions for use.

16. A method to immunize a subject against a viral infection comprising, or consisting essentially of, or yet further consisting of administering to a subject in need thereof an effective amount of the composition of claim 1 and wherein the subject is optionally a mammal or a human subject, further optionally an infant or juvenile.

17. The method of claim 16, where the vaccine is administered in one or more doses.

18. A method to prepare a vaccine formulation, comprising admixing:
a. an effective amount of a virus or viral antigen;
b. from about 2% to about 10% of a bulking agent;
c. from about 5 mM to about 25 mM of a buffer;
d. from about 0.5% to about 1.5% of a sugar composition; and
e. from about 0.1% to about 10% or from about 0.5 mM to about 1.5 mM of a stabilizer.

19. The method of claim 18, further comprising from about 0.005% to about 1.5% of a surfactant solution.

20. A kit comprising one or more of the vaccine formulation of claim 12, and instructions for use.
